(19) United States
(12) Patent Application Publication
(10) Pub. No.: US 2004/0018557 A1
(43) Pub. Date: Jan. 29, 2004

(54) Bispecific antibody point mutations for enhancing rate of clearance

(75) Inventors: Zhengxing Qu, Warren, NJ (US); Hans Hansen, Picayune, MS (US); David Goldenberg, Mendham, NJ (US)

Correspondence Address: FOLEY AND LARDNER, SUITE 500, 3000 K STREET NW, WASHINGTON, DC, 20007, US

(73) Assignee: Immunomedics, Inc.

(21) Appl. No.: 10/377,109

(22) Filed: Mar. 3, 2003

Related U.S. Application Data
(60) Provisional application No. 60/361,037, filed on Mar. 1, 2002.

(51) Int. Cl. G01N 33/53; A61K 39/395; C07K 16/44

(52) U.S. Cl. 435/7.1; 530/387.3; 530/388.15; 424/132.1

(57) ABSTRACT

A mutant bispecific antibody that includes (a) a human hinge constant region from IgG having one or more amino acid mutations in the C_{H}2 domain, (b) two scFvs; and (c) two Fvs has been constructed. This type of antibody displays enhanced clearance, which has been found to be particularly useful in the context of pre-targeting methods.

This application claims priority to U.S. Provisional Application No. 60/361,037, which is incorporated herein by reference in its entirety

### BACKGROUND OF THE INVENTION

1. Field of the Invention

The present invention relates to a mutant bispecific antibody (bsAb) which clears from a patient's body faster than the corresponding parent bsAb. In particular, the invention relates to a mutant bsAb, containing a human hinge constant region from IgG, two scFvs and two Fvs, wherein the hinge constant region contains one or more amino acid mutations in the C_{H}2-C_{H}3 domain interface region.

2. Related Art

The detection of a target site benefits from a high signal-to-background ratio of a detection agent. Therapy benefits from as high an absolute accretion of therapeutic agent at the target site as possible, as well as a reasonably long duration of uptake and binding. In order to improve the targeting ratio and amount of agent delivered to a target site, the use of targeting vectors comprising diagnostic or therapeutic agents conjugated to a targeting moiety for preferential localization has long been known.

Examples of targeting vectors include diagnostic or therapeutic agent conjugates of targeting moieties such as antibody or antibody fragments, cell- or tissue-specific peptides, and hormones and other receptor-binding molecules. For example, antibodies against different determinants associated with pathological and normal cells, as well as associated with pathogenic microorganisms, have been used for the detection and treatment of a wide variety of pathological conditions or lesions. In these methods, the targeting antibody is directly conjugated to an appropriate detecting or therapeutic agent as described, for example, in Hansen et al., U.S. Pat. No. 3,927,193 and Goldenberg, U.S. Pat. Nos. 4,331,647, 4,348,376, 4,361,544, 4,468,457, 4,444,744, 4,460,459, 4,460,561, 4,624,846 and 4,818,709, the disclosures of all of which are incorporated in their entirety herein by reference.

One problem encountered in direct targeting methods, i.e., in methods wherein the diagnostic or therapeutic agent (the “active agent”) is conjugated directly to the targeting moiety, is that a relatively small fraction of the conjugate actually binds to the target site, while the majority of conjugate remains in circulation and compromises in one way or another the function of the targeted conjugate. In the case of a diagnostic conjugate, for example, a radioimmunoscintigraphic or magnetic resonance imaging conjugate, non-targeted conjugate which remains in circulation can increase background and decrease resolution. In the case of a therapeutic conjugate having a very toxic therapeutic agent, e.g., a radioisotope, drug or toxin, attached to a long-circulating targeting moiety, such as an antibody, circulating conjugate can result in unacceptable toxicity to the host, such as marrow toxicity or systemic side effects.

Pretargeting methods have been developed to increase the target:background ratios of the detection or therapeutic agents. Examples of pre-targeting and biotin/avidin approaches are described, for example, in Goodwin et al., U.S. Pat. No. 4,863,713; Goodwin et al., J. Nucl. Med. 29:226, 1988; Hnatowich et al., J. Nucl. Med. 28:1294, 1987; Oehr et al., J. Nucl. Med. 29:728, 1988; Klibanov et al., J. Nucl. Med. 29:1951, 1988; Sinitsyn et al., J. Nucl. Med. 30:66, 1989; Kalofonos et al., J. Nucl. Med. 31:1791, 1990; Schechter et al., Int. J. Cancer 48:167, 1991; Paganelli et al., Cancer Res. 51:5960, 1991; Paganelli et al., Nucl. Med. Commun. 12:211, 1991; U.S. Pat. No. 5,256,395; Stickney et al., Cancer Res. 51:6650, 1991; Yuan et al., Cancer Res. 51:3119, 1991; U.S. Pat. No. 6,077,499; U.S. Ser. No. 09/597,580; U.S. Ser. No. 10/361,026; U.S. Ser. No. 09/337,756; U.S. Ser. No. 09/823,746; U.S. Ser. No. 10/116,116; U.S. Ser. No. 09/382,186; U.S. Ser. No. 10/150,654; U.S. Pat. No. 6,090,381; U.S. Pat. No. 6,472,511; U.S. Ser. No. 10/114,315; U.S. Provisional Application No. 60/386,411; U.S. Provisional Application No. 60/345,641; U.S. Provisional Application No. 60/3328,835; U.S. Provisional Application No. 60/426,379; U.S. Ser. No. 09/823,746; U.S. Ser. No. 09/337,756; and U.S. Provisional Application No. 60/342,103 all of which are incorporated by reference herein in their entirety.

In pretargeting methods, a primary targeting species (which is not bound to a diagnostic or therapeutic agent) is administered. The primary targeting species comprises a targeting moiety which binds to the target site and a binding moiety which is available for binding to a binding site on a targetable construct. Once sufficient accretion of the primary targeting species is achieved, a targetable construct is administered. The targetable construct comprises a binding site which recognizes the available binding site of the primary targeting species and a diagnostic or therapeutic agent.

Pretargeting is an approach which offers certain advantages over the use of direct targeting methods. For example, use of the pretargeting approach for the in vivo delivery of radionuclides to a target site for therapy, e.g., radioimmunotherapy, reduces the marrow toxicity caused by prolonged circulation of a radioimmunoconjugate. This is because the radioisotope is delivered as a rapidly clearing, low molecular weight chelate rather than directly conjugated to a primary targeting molecule, which is often a long-circulating species.

A problem encountered with pretargeting methods is that circulating primary targeting species (primary targeting species which is not bound to the target site) interferes with the binding of the targetable conjugate to targeting species that are bound to the target site (via the binding moiety on the primary targeting species). Thus, there is a need for methods of minimizing the amount of circulating primary targeting species.

Some attempts have been made to minimize the amount of circulating primary targeting species. One method for obtaining this goal is to prepare a primary targeting species with an accelerated rate of clearance from the body. For example, Ward et al. (U.S. Pat. No. 6,165,745) has synthesized a mutant IgG1 from murine and Hornick et al. The Journal of Nuclear Medicine 11 355-362 (2000) has synthesized a mutant chimeric TNT-3 antibody. These mutant antibodies differ from the mutant bsAb of the present invention. One difference is that the inventive mutant bsAb of the present invention is a bispecific antibody, whereas the antibodies of Hornick et al. and Ward et al. are monospecific antibodies. This difference is significant because a bispecific antibody has different properties than a monospecific antibody. Another difference between the present mutant bsAb and the murine antibody of Ward et al. is that the murine antibody of Ward et al. does not have an effector function. Therefore, the antibody of Ward et al. is not capable of fixing complement or effecting ADCC (antibody dependent cell cytotoxicity), as is the present mutant bsAb.

### SUMMARY OF THE INVENTION

It is an object of the invention to provide a mutant bsAb, containing a human hinge constant region from IgG, two scFvs and two Fvs, wherein the hinge constant region contains one or more amino acid mutations in the C_{H}2-C_{H}3 domain interface region. In some embodiments, the Fvs and scFvs are CDR-grafted murine or humanized components. In other embodiments, the Fvs and scFvs are human or humanized components. In some embodiments, the hinge constant region contains a mutation of isoleucine 253 to alanine. The present invention also provides a mutant bsAb wherein the Fvs are derived from hMN14-IgG, a humanized Class III, anti-CEA mAb (see U.S. Pat. No. 5,874,540) the scFvs are 734scFv and isoleucine at position 253 in the hinge constant region is mutated to alanine.

### BRIEF DESCRIPTION OF THE DRAWINGS

FIG. 1 shows the heavy chain cDNA and amino acid sequences of hMN-14. The V_{H}, C_{H}1, Hinge, C_{H}2 and C_{H}3 regions are shown. The isoleucine at amino acid position 274 corresponds to isoleucine 253 according to the numbering system of Edelman, et al. See Edelman et al. Biochemistry 63, 78-85 (1969).

FIG. 2 shows the light chain cDNA and amino acid sequences of hMN-14. The Vκ and Cκ regions are shown.

FIG. 3 shows the biodistribution of hMN-14IgG^{I253A}-(734scFv)₂ in human colonic tumor-bearing mice, 1, 2, 3 and 4 days post injection. The designation “I253A” means that the isoleucine at position 253 is changed to an alanine. Data were expressed as a median percentage of injected dose per gram (% ID/g).

FIG. 4 shows the biodistribution of hMN-14IgG-(734scFv)₂ in human colonic tumor-bearing mice, 1, 2, 3 and 4 days post injection. Data were expressed as a median percentage of injected dose per gram (% ID/g).

FIG. 5 shows biodistribution data obtained from pretargeting experiments involving ¹²⁵I-hMN-14IgG-(734scFv)₂. The targetable construct was Tc-99m-labeled di-DTPA, IMP-192. Human colonic tumor-bearing mice were pretargeted with ¹²⁵I-hMN-14IgG-(734scFv)₂ for four days after which they were injected with a targetable conjugate. Data were obtained 3, 6 and 24 hours post injection of the targetable conjugate. Data are expressed as a median percentage of injected dose per gram (% ID/g). The tumor-to-blood ratio is reported under the entry for “Blood”. The left side of the chart shows data for ¹²⁵I-labeled bsAb and the right side of the chart shows data for ^{99m}Tc-labeled targetable construct.

FIG. 6 shows biodistribution data obtained from pretargeting experiments involving ¹²⁵I-hMN-14IgG-(734scFv)₂. The targetable construct was Tc-99m-labeled di-DTPA, IMP-192. Human colonic tumor-bearing mice were pretargeted with ¹²⁵I-hMN-14IgG-(734scFv)₂ for six days after which they were injected with a targetable conjugate. Data were obtained 3, 6 and 24 hours post injection of the targetable conjugate. Data are expressed as a median percentage of injected dose per gram (% ID/g). The tumor-to-blood ratio is reported under the entry for “Blood”. The left side of the chart shows data for ¹²⁵I-labeled bsAb and the right side of the chart shows data for ^{99m}Tc-labeled targetable construct.

FIG. 7 shows biodistribution data obtained from pretargeting experiments involving ¹²⁵I-hMN-14IgG^{I253A}-(734scFV)₂. The targetable construct was Tc-99m-labeled di-DTPA, IMP-192. Human colonic tumor-bearing mice were pretargeted with ¹²⁵I-hMN-14IgG^{I253A}-(734scFv)₂ for four days after which they were injected with a targetable conjugate. Data were obtained 3, 6 and 24 hours post injection of the targetable conjugate. Data are expressed as a median percentage of injected dose per gram (% ID/g). The tumor-to-blood ratio is reported under the entry for “Blood”. The left side of the chart shows data for ¹²⁵I-labeled bsAb and the right side of the chart shows data for ^{99m}Tc-labeled targetable construct.

FIG. 8 shows an ellution profile of a known standard of hMN-14IgG^{I253A}-(734scFv)₂ on a Bio-Sil SEC 250 300 mm×7.8 mm HPLC column elluted at 1 mL/min with 0.2 M phosphate buffer pH 6.8.

FIG. 9 shows an ellution profile of a known standard of Tc-99m IMP 192 on a Bio-Sil SEC 250 300 mm×7.8 mm HPLC column elluted at 1 mL/min with 0.2 M phosphate buffer pH 6.8.

FIG. 10 shows an ellution profile of a 1:1 mixture of hMN-14IgG^{I253A}-(734scFv)₂ to Tc-99m IMP 192 on a Bio-Sil SEC 250 300 mm×7.8 mm HPLC column elluted at 1 mL/min with 0.2 M phosphate buffer pH 6.8.

FIG. 11 shows an ellution profile of a 1:5 mixture of hMN-14IgG^{I253}A-(734scFv)₂ to Tc-99m IMP 192 on a Bio-Sil SEC 250 300 mm×7.8 mm HPLC column elluted at 1 mL/min with 0.2 M phosphate buffer pH 6.8.

FIG. 12 shows an ellution profile of a 20:1 mixture of hMN-14IgG^{I253A}-(734scFv)₂ to Tc-99m IMP 192 on a Bio-Sil SEC 250 300 mm×7.8 mm HPLC column elluted at 1 mL/min with 0.2 M phosphate buffer pH 6.8.

### DETAILED DESCRIPTION OF THE PREFERRED EMBODIMENTS

Unless otherwise specified, the terms “a” or “an” mean “one or more”.

I. Overview

The present invention relates to a mutant bsAb containing a human hinge constant region from IgG, two scFvs and two Fvs, wherein the hinge constant region contains one or more amino acid mutations in the C_{H}2-C_{H}3 domain interface region. The mutant bsAb of the present invention clears a patient's body at a faster rate than the corresponding parent bsAb. Bispecific antibodies are disclosed in U.S. application Ser. No. 09/337,756, filed Jun. 22, 1999. When employed in a pretargeting method, the amount of circulating primary targeting species (mutant bsAb not bound to the target site) is minimized. Additionally, the amount of targetable construct trapped in the blood is minimized.

The human hinge constant region may contain an effector function. The Fc portion of the antibody molecule provides effector functions, such as complement fixation and ADCC (antibody dependent cell cytotoxicity), which set mechanisms into action that may result in cell lysis. However, it is possible that the Fc portion is not required for therapeutic function, with other mechanisms, such as apoptosis, coming into play. Therefore, innate ADCC, apoptosis induction and complement activation/lysis may be achieved.

The scFvs are specific for a binding site on a targetable construct. The targetable construct is comprised of a carrier portion and at least 1 unit of a recognizable hapten. Examples of recognizable haptens include, but are not limited to, histamine succinyl glycine (HSG), DTPA and fluorescein isothiocyanate. The targetable construct may be conjugated to a variety of agents useful for treating or identifying diseased tissue. Examples of conjugated agents include, but are not limited to, chelators, metal chelate complexes, drugs, toxins (e.g., ricin, abrin, ribonuclease, DNase I, Staphylococcal enterotoxin-A, pokeweed antiviral protein, gelonin, diphtherin toxin, Pseudomonas exotoxin, Pseudomonas endotoxin) and other effector molecules. Suitable drugs for conjugation include doxorubicin analogs, SN-38, etoposide, methotrexate, 6-mercaptopurine or etoposide phosphate, calicheamicin, paclitaxel, 2-pyrrolinodoxorubicin, CC-1067, and adozelesin or a combination thereof. Exemplary drugs are nitrogen mustards, ethylenimine derivatives, alkyl sulfonates, nitrosoureas, triazenes, folic acid analogs, anthracyclines, taxanes, COX-2 inhibitors, pyrimidine analogs, purine analogs, antibiotics, enzymes, epipodophyllotoxins, platinum coordination complexes, vinca alkaloids, substituted ureas, methyl hydrazine derivatives, adrenocortical suppressants, antagonists, endostatin, taxols, camptothecins, doxorubicins and their analogs, and a combination thereof. Additionally, enzymes useful for activating a prodrug or increasing the target-specific toxicity of a drug can be conjugated to the targetable construct. Thus, the use of a mutant bsAb containing scFvs which are reactive to a targetable construct allows a variety of therapeutic and diagnostic applications to be performed without raising new bsAbs for each application.

Additionally, the present invention encompasses a method for detecting or treating target cells, tissues or pathogens in a mammal, comprising administering an effective amount of a mutant bsAb comprising a human hinge constant region from IgG, two Fvs and two scFvs, wherein the hinge constant region contains one or more amino acid mutations in the C_{H}2-C_{H}3 domain interface region. As used herein, the term “pathogen” includes, but is not limited to fungi (e.g. Histoplasma capsulatum, Blastomyces dermatitidis, Coccidioides immitis, and species of Candida), viruses (e.g., human immunodeficiency virus (HIV), herpes virus, cytomegalovirus, rabies virus, influenza virus, hepatitis B virus, Sendai virus, feline leukemia virus, Reo virus, polio virus, human serum parvo-like virus, simian virus 40, respiratory syncytial virus, mouse mammary tumor virus, Varicella-Zoster virus, Dengue virus, rubella virus, measles virus, adenovirus, human T-cell leukemia viruses, Epstein-Barr virus, murine leukemia virus, mumps virus, vesicular stomatitis virus, Sindbis virus, lymphocytic choriomeningitis virus, wart virus and blue tongue virus), parasites, microbes (e.g. rickettsia) and bacteria (e.g., Streptococcus agalactiae, Legionella pneumophilia, Streptococcus pyogenes, Escherichia coli, Neisseria gonorrhoeae, Neisseria meningitidis, Pneumococcus, Hemophilis influenzae B, Treponema pallidum, Lyme disease spirochetes, Pseudomonas aeruginosa, Mycobacterium leprae, Brucella abortus, Mycobacterium tuberculosis, Anthrax spores and Tetanus toxin). See U.S. Pat. No. 5,332,567.

As used herein, the term “antibody” refers to a full-length (i.e., naturally occurring or formed by normal immunoglobulin gene fragment recombinatorial processes) immunoglobulin molecule (e.g., an IgG antibody) or an immunologically active (i.e., specifically binding) portion of an immunoglobulin molecule, like an antibody fragment. The term antibody encompasses chimeric, cdr-grafted (humanized), and fully human antibodies. The term “IgG” is used to mean an antibody, i.e., an immunoglobulin G, generated against, and capable of binding specifically to an antigen. The term antibody is abbreviated as Ab. A monoclonal antibody is abbreviated as mAb.

A human antibody is an antibody obtained from transgenic mice that have been “engineered” to produce specific human antibodies in response to antigenic challenge. In this technique, elements of the human heavy and light chain locus are introduced into strains of mice derived from embryonic stem cell lines that contain targeted disruptions of the endogenous heavy chain and light chain loci. The transgenic mice can synthesize human antibodies specific for human antigens, and the mice can be used to produce human antibody-secreting hybridomas. Methods for obtaining human antibodies from transgenic mice are described by Green et al., Nature Genet. 7:13 (1994), Lonberg et al., Nature 368:856 (1994), and Taylor et al., Int. Immun. 6:579 (1994). A fully human antibody also can be constructed by genetic or chromosomal transfection methods, as well as phage display technology, all of which are known in the art. See for example, McCafferty et al., Nature 348:552-553 (1990) for the production of human antibodies and fragments thereof in vitro, from immunoglobulin variable domain gene repertoires from unimmunized donors. In this technique, antibody variable domain genes are cloned in-frame into either a major or minor coat protein gene of a filamentous bacteriophage, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. In this way, the phage mimics some of the properties of the B cell. Phage display can be performed in a variety of formats, for their review, see e.g. Johnson and Chiswell, Current Opinion in Structural Biology 3:5564-571 (1993).

Human antibodies may also be generated by in vitro activated B cells. See U.S. Pat. Nos. 5,567,610 and 5,229,275, which are incoporated in their entirety by reference.

An antibody fragment is a portion of an antibody such as F(ab′)₂, F(ab)₂, Fab′, Fab, Fv, scFv and the like. Regardless of structure, an antibody fragment binds with the same antigen that is recognized by the intact antibody. For example, an anti-CEA monoclonal antibody fragment binds with an epitope of CEA.

The term “antibody fragment” also includes any synthetic or genetically engineered protein that acts like an antibody by binding to a specific antigen to form a complex. For example, antibody fragments include isolated fragments consisting of the light chain variable region, “Fv” fragments consisting of the variable regions of the heavy and light chains, recombinant single chain polypeptide molecules in which light and heavy variable regions are connected by a peptide linker (“scFv proteins”), and minimal recognition units consisting of the amino acid residues that mimic the hypervariable region.

A chimeric antibody is a recombinant protein that contains the variable domains and complementary determining regions derived from a first species, such as a rodent antibody, while the heavy and light chain constant regions of the antibody molecule is derived from a second species, such as a human antibody.

Humanized antibodies are recombinant proteins in which the complementarity determining regions of a monoclonal antibody have been transferred from heavy and light variable chains of a first species immunoglobulin, such as a murine immunoglobulin into the human heavy and light variable domains while the heavy and light chain constant regions of the antibody molecule is derived from a human antibody. Humanized antibodies are also referred to as CDR-grafted antibodies.

As used herein, the term “bispecific antibody” is an antibody capable of binding to two different moieties, i.e., a targeted tissue and a targetable construct.

As used herein, a therapeutic agent is a molecule or atom which is administered to a subject in combination according to a specific dosing schedule with the antibody of the present invention or conjugated to an antibody moiety to produce a conjugate which is useful for therapy. Examples of therapeutic agents include drugs, toxins, hormones, enzymes, immunomodulators, chelators, boron compounds, photoactive agents or dyes, and radioisotopes. Exemplary immunomodulators may be selected from the group consisting of a cytokine, a stem cell growth factor, a lymphotoxin, a hematopoietic factor, a colony stimulating factor (CSF), an interferon (IFN), erythropoietin, thrombopoietin and a combination thereof. Specifically useful are lymphotoxins, such as tumor necrosis factor (TNF), hematopoietic factors, such as interleukin (IL), colony stimulating factor, such as granulocyte-colony stimulating factor (G-CSF) or granulocyte macrophage-colony stimulating factor (GM-CSF)), interferon, such as interferons-α, -β or -γ, and stem cell growth factor, such as designated “S1 factor”. More specifically, immunomodulator, such as IL-1, IL-2, IL-3, IL-6, IL-10, IL-12, IL-18, interferon-γ, TNF-α or a combination thereof are useful in the present invention. The term “scFv” is used to mean recombinant single chain polypeptide molecules in which light and heavy chain variable regions of an antibody are connected by a peptide linker.

The term “Fv” is used to mean fragments consisting of the variable regions of the heavy and light chains.

A “recombinant host” may be any prokaryotic or eukaryotic cell that contains either a cloning vector or expression vector. This term also includes those prokaryotic or eukaryotic cells, as well as an transgenic animal, that have been genetically engineered to contain the cloned gene(s) in the chromosome or genome of the host cell or cells of the host cells. Suitable mammalian host cells include myeloma cells, such as SP2/0 cells, and NS0 cells, as well as Chinese Hamster Ovary (CHO) cells, hybridoma cell lines and other mammalian host cell useful for expressing antibodies. Also particularly useful to express mAbs and other fusion proteins is a human cell line, PER.C6 disclosed in WO 0063403 A2, which produces 2 to 200-fold more recombinant protein as compared to conventional mammalian cell lines, such as CHO, COS, Vero, Hela, BHK and SP2-cell lines. Special transgenic animals with a modified immune system are particularly useful for making fully human antibodies.

The antigen may be any antigen. An exemplary antigen is a cell surface or tumor-associated antigen, or an antigen associated with a microorganism or parasite, or with a diseased tissue or cell type leading to disease, such as a B- or T-cell involved in autoimmune disease, or a target antigen of a cardiovascular or neurological disease (e.g., atherosclerotic plaque or embolus in the former and amyloid in the latter, such as associated with Alzheimer's disease). As used herein, the term “tissue” is used to mean a tissue as one of ordinary skill in the art would understand it to mean. As envisioned in the current application, tissue is also used to mean individual or groups of cells, or cell cultures, of a bodily tissue or fluid (e.g., blood cells). Furthermore, the tissue may be within a subject, or biopsied or removed from a subject. The tissue may also be a whole or any portion of a bodily organ. Additionally, the tissue may be “fresh” in that the tissue would be recently removed from a subject without any preservation steps between the excision and the methods of the current invention. The tissue may also have been preserved by such standard tissue preparation techniques including, but not limited to, freezing, quick freezing, paraffin embedding and tissue fixation, prior to application of the methods of the current invention.

A “targeted tissue” is a system, organ, tissue, cell, receptor or organelle to which a targetable conjugate may be delivered. In the therapeutic aspects of the invention, the targeted tissue is infected, dysfunctional, displaced or ectopic (e.g., infected cells, cancer cells, endometriosis, etc.). Normal tissues, such as bone marrow, may also be ablated, as needed in a therapeutic intervention, by these methods. In diagnostic aspects of the invention, it is desired to detect the targeted tissue.

As used herein, the term “subject” refers to any animal (i.e., vertebrates and invertebrates) including, but not limited to humans and other primates, rodents (e.g., mice, rats, and guinea pigs), lagamorphs (e.g., rabbits), bovines (e.g, cattle), ovines (e.g., sheep), caprines (e.g., goats), porcines (e.g., swine), equines (e.g., horses), canines (e.g., dogs), felines (e.g., cats), domestic fowl (e.g., chickens, turkeys, ducks, geese, other gallinaceous birds, etc.), as well as feral or wild animals, including, but not limited to, such animals as ungulates (e.g., deer), bear, fish, lagamorphs, rodents, birds, etc. It is not intended that the term be limited to a particular age or sex. Thus, adult and newborn subjects, as well as fetuses, whether male or female, are encompassed by the term.

As used herein, the term “parent bsAb” is used to mean a bsAb which is similar to a mutant bsAb in every way except that the hinge constant region of the parent bsAb does not contain one or more amino acid mutations in the C_{H}2-C_{H}3 domain interface region.

As used herein, the term “hinge constant region” comprises the C₁, C_{H}1, hinge, C_{H}2 and C_{H}3 regions of an IgG. The heavy chain constant region comprises the C_{H}1, hinge, C_{H}2 and C_{H}3 regions, while the light chain constant region comprises the C₁ region.

II. The Mutant Bispecific Antibody

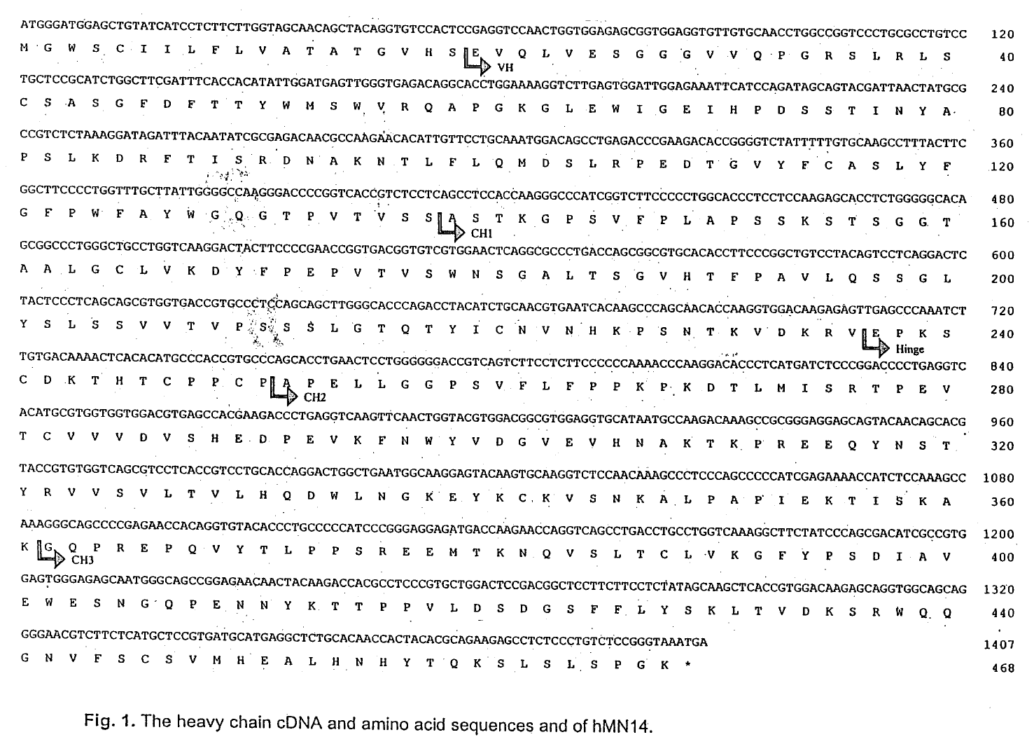

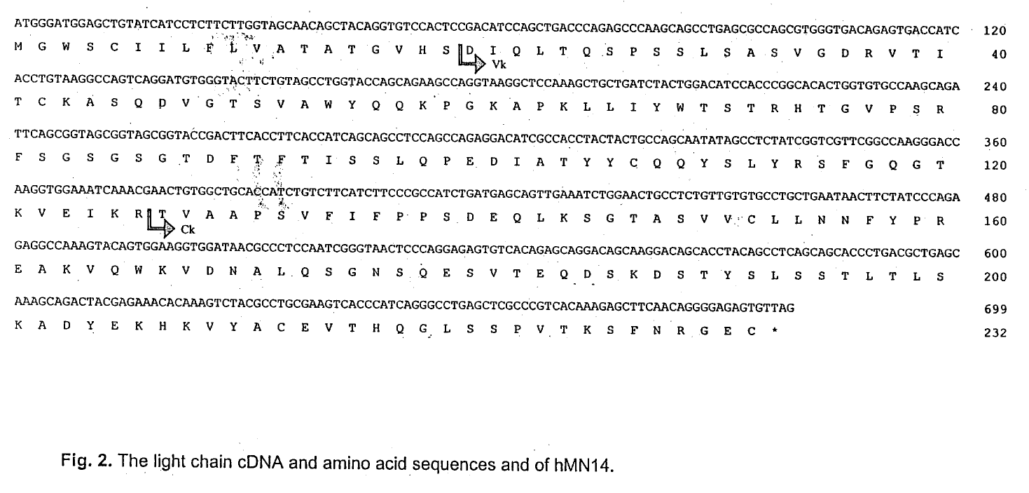

The Fvs of the mutant bsAb are derived from an antibody and specifically bind a targeted tissue. Exemplary Fvs are derived from anti-CD20 antibodies, such as those described in Provisional U.S. Application titled “Anti-CD20 Antibodies And Fusion Proteins Thereof And Methods Of Use”, Attorney Docket No. 18733/1073, U.S. Provisional No. 60/356,132, U.S. Provisional Application No. 60/416,232 and Attorney Docket No. 18733/1155 (the contents of which are in their entirety herein by reference); hMN-14 antibodies, such as those disclosed in U.S. Pat. No. 5,874,540 (the contents of which are incorporated in their entirety herein by reference), which is a Class III anti-carcinoembryonic antigen antibody (anti-CEA antibody); Mu-9 antibodies, such as those described in U.S. application Ser. No. 10/116,116 (the contents of which are incorporated in their entirety herein by reference); LL1 antibodies, such as those described in U.S. Provisional Application No. 60/360,259 (the contents of which are incorporated in their entirety herein by reference); AFP antibodies, such as those described in U.S. Provisional Application No. 60/399,707 (the contents of which are incorporated in their entirety herein by reference); PAM4 antibodies, such as those described in Provisional U.S. Application titled “Monoclonal Antibody cPAM4”, Attorney Docket No. 18733/1102 (the contents of which are incorporated in their entirety herein by reference); RS7 antibodies, such as those described in U.S. Provisional Application No. 60/360,229 (the contents of which are incorporated in their entirety herein by reference); and CD22 antibodies, such as those disclosed in U.S. Pat. Nos. 5,789,554 and 6,187,287 and U.S. application Ser. Nos. 09/741,843 and 09/988,013 (the contents of which are incorporated in their entirety herein by reference). Many other tumor-associated antigens of hematopoietic and solid tumors are known to those skilled in the art, as contained in the referenced applications, and include (but are not limited to) CD15, CD19, CD20, CD21, CD22, CD23, CD25, CD40, CD45, CD66, CD74, CD80, Ii, Ia, HLA-DR, PSMA, PSA, prostastic acid phosphatase, tenascin, Le(y), AFP, HCG, CEA, CSAp, PAM4, MUC1, MUC2, MUC3, MUC4, EGP-1, EGP-2, EGFR, HER2/neu, insulin growth-factor receptors, S100, VEGF, Placenta Growth Factor (P1GF), placental alkaline phosphatase, necrosis products, oncogene products, and the like. The heavy chain cDNA and amino acid sequences of hMN-14 are shown in FIG. 1 and the light chain cDNA and amino acid sequences of hMN-14 are shown in FIG. 2.

The cDNA encoding the Fvs may be inserted into a vector encoding the hinge constant region. An exemplary expression vector, pdHL2, which encodes the amino acids of the hinge constant region of IgG1 was reported by Gillies S.D., Lo KM, and Wesolowski, J. J. Immunol Methods 125 191-202 (1989) and Losman, M. J. et al. Cancer Supplement 80 2660-2666 (1997) and may be used to construct mutant bispecific antibodies of the present invention.

The Fvs can be from murine antibodies, cdr-grafted (humanized) antibodies, or human antibodies. The Fvs can be derived from human monoclonal antibodies, transgenic mice with human Fv-libraries, or phage/ribosome human IgG libraries.

When the Fvs are derived from CDR-grafted antibodies, appropriate variable region framework sequences may be used having regard to the class or type of the donor antibody from which the antigen binding regions are derived. Preferably, the type of human framework used is of the same or similar class or type as the donor antibody. Advantageously, the framework is chosen to maximize or optimize homology with the donor antibody sequence, particularly at positions spatially close to or adjacent the CDRs. Examples of human frameworks which may be used to construct CDR-grafted antibodies are LAY, POM, TUR, TEI, KOL, NEWM, REI and EU (Kabat et al, 1987). KOL and NEWM and are suitable for heavy chain construction. REI is suitable for light chain construction and EU is suitable for both heavy chain and light chain construction.

The light or heavy chain variable regions of the CDR-grafted antibodies may be fused to human light or heavy chain constant domains as appropriate (the term “heavy chain constant domains” as used herein is to be understood to include hinge regions unless specified otherwise). The human constant domains of the CDR-grafted antibodies, where present, may be selected having regard to the proposed function of the antibody, in particular, the effector functions which may be required. For example, IgG1 and IgG3 isotype domains may be used when the CDR-grafted antibody is intended for therapeutic purposes and antibody effector functions are required. Alternatively, IgG2 and IgG4 isotype domains may be used when the CDR-grafted antibody is intended for purposes for which antibody effector functions are not required, e.g., for imaging, diagnostic or cytotoxic targeting purposes. Light chain human constant domains which may be fused to the light chain variable region include human Lambda or, especially, human Kappa chains.

The hinge constant region of the bi-specific mutant antibody contains one or more amino acid mutations in the C_{H}2-C_{H}3 domain interface region. In other words, when the human hinge constant region of the bi-specific mutant antibody is compared to the human hinge constant region of the bi-specific parent antibody, the regions will differ by one or more amino acids.

A mutation may encompass, for example, a “conservative” change, wherein a substituted amino has similar structural or chemical properties, such as charge or size (e.g., replacement of leucine with isoleucine). A mutation also encompasses, for example, a “non-conservative” change (e.g., replacement of a glycine with a tryptophan).

In preferred embodiments, the amino acid at position 253 (according to the numbering system of Edelman) is mutated. An exemplary mutation at this position replacing isoleucine with alanine. In some embodiments, the amino acid at position 253 is mutated to an amino acid wherein the pharmacokinetics of clearance of the mutant bsAb are similar to that observed when the amino acid at position 253 is changed to alanine.

In one embodiment, the hinge constant region of the bi-specific mutant antibody comprises the amino acid sequences of human IgG1. The amino acids encoding the C_{H}1, hinge, C_{H}2 and C_{H}3 regions of the heavy chain are shown as amino acid numbers 139-468 of FIG. 1, while the amino acids encoding the C₁ chain are shown as amino acid numbers 128-232 of FIG. 2. It is noted that the numbering system used to identify isoleucine 253 is consistent with the numbering system used by Edelman et al. in their disclosure of the Eu heavy and light chains. Edelman et al. Biochemistry 63, 78-85 (1969).

The scFv component of the bi-specific mutant antibody specifically binds a targetable construct. The use of any scFv component is contemplated by the present invention. Preferred scFv components are 679 scFv (derived from a murine anti-HSG) and 734scFv (derived from a murine anti-diDTPA). The scFv can be murine, cdr-grafted (humanized) or human.

When the scFvs are derived from CDR-grafted antibodies, appropriate variable region framework sequences may be used having regard to the class or type of the donor antibody from which the antigen binding regions are derived. Preferably, the type of human framework used is of the same or similar class or type as the donor antibody. Advantageously, the framework is chosen to maximize or optimize homology with the donor antibody sequence, particularly at positions spatially close to or adjacent the CDRs. Examples of human frameworks which may be used to construct CDR-grafted antibodies are LAY, POM, TUR, TEI, KOL, NEWM, REI and EU (Kabat et al, 1987). KOL and NEWM and are suitable for heavy chain construction. REI is suitable for light chain construction and EU is suitable for both heavy chain and light chain construction.

The light or heavy chain variable regions of the CDR-grafted antibodies may be fused to human light or heavy chain constant domains as appropriate, (the term “heavy chain constant domains” as used herein are to be understood to include hinge regions unless specified otherwise). The human constant domains of the CDR-grafted antibodies, where present, may be selected having regard to the proposed function of the antibody, in particular the effector functions which may be required. For example, IgG1 and IgG3 isotype domains may be used when the CDR-grafted antibody is intended for therapeutic purposes and antibody effector functions are required. Alternatively, IgG2 and IgG4 isotype domains may be used when the CDR-grafted antibody is intended for purposes for which antibody effector functions are not required, e.g. for imaging, diagnostic or cytotoxic targeting purposes. Light chain human constant domains which may be fused to the light chain variable region include human Lambda or, especially, human Kappa chains.

A preferred mutant bsAb is hMN-14IgG^{I253A}-(734scFv)₂. In this mutant bsAb, the FVs are derived from hMN-14IgG, the scFvs are 734scFV (derived from a murine anti-diDTPA) and the hinge constant region comprises the amino acid sequences of human IgG1.

In an embodiment of the present invention, a one to one binding interaction is obtained between the mutant bsAb and a targetable construct. For example, when the mutant bsAb of the present invention interacts with the bivalent targetable construct IMP 192 which contains two DTPA sites, one bsAb binds to one IMP 192. This interaction is illustrated by Example 3.

III. Constructs Targetable to the Mutant bsAb

In some embodiments, the mutant bsAb of the present invention binds a targetable construct. Preferably, the scFvs of the mutant bsAb bind the targetable construct. The targetable construct can be of diverse structure, but is selected not only to elicit sufficient immune responses, but also for rapid in vivo clearance. Exemplary targetable constructs for use in the present application are described in U.S. application Ser. No. 09/337,756 filed Jun. 22, 1999 and in U.S. application Ser. No. 09/823,746, filed Apr. 3, 2001, the entire contents of which are incorporated herein by reference.

Hydrophobic agents are best at eliciting strong immune responses, whereas hydrophilic agents are preferred for rapid in vivo clearance, thus, a balance between hydrophobic and hydrophilic needs to be established. This is accomplished, in part, by relying on the use of hydrophilic chelating agents to offset the inherent hydrophobicity of many organic moieties. Also, sub-units of the targetable construct may be chosen which have opposite solution properties, for example, peptides, which contain amino acids, some of which are hydrophobic and some of which are hydrophilic. Aside from peptides, carbohydrates may be used.

Peptides having as few as two amino-acid residues may be used, preferably two to ten residues, if also coupled to other moieties, such as chelating agents. The linker should be a low molecular weight conjugate, preferably having a molecular weight of less than 50,000 daltons, and advantageously less than about 20,000 daltons, 10,000 daltons or 5,000 daltons, including the metal ions in the chelates. For instance, the known peptide DTPA-Tyr-Lys(DTPA)-OH (wherein DTPA is diethylenetriaminepentaacetic acid) has been used to generate antibodies against the indium-DTPA portion of the molecule. However, by use of the non-indium-containing molecule, and appropriate screening steps, new Abs against the tyrosyl-lysine dipeptide can be made. More usually, the antigenic peptide will have four or more residues, such as the peptide DOTA-Phe-Lys(HSG)-Tyr-Lys(HSG)-NH₂, wherein DOTA is 1,4,7,10-tetraazacyclododecanetetraacetic acid and HSG is the histamine succinyl glycyl group of the formula:

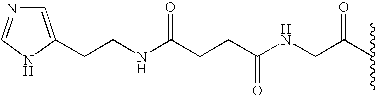

The non-metal-containing peptide may be used as an immunogen, with resultant Abs screened for reactivity against the Phe-Lys-Tyr-Lys backbone.

The invention also contemplates the incorporation of unnatural amino acids, e.g., D-amino acids, into the backbone structure to ensure that, when used with the final bsAb/linker system, the scFv component which recognizes the linker moiety is completely specific. The invention further contemplates other backbone structures such as those constructed from non-natural amino acids and peptoids.

The peptides to be used as immunogens are synthesized conveniently on an automated peptide synthesizer using a solid-phase support and standard techniques of repetitive orthogonal deprotection and coupling. Free amino groups in the peptide, which are to be used later for chelate conjugation, are advantageously blocked with standard protecting groups such as an acetyl group. Such protecting groups will be known to the skilled artisan. See Greene and Wuts Protective Groups in Organic Synthesis, 1999 (John Wiley and Sons, N.Y.). When the peptides are prepared for later use the mutant bsAb, they are advantageously cleaved from the resins to generate the corresponding C-terminal amides, in order to inhibit in vivo carboxypeptidase activity.

The haptens of the immunogen comprise an immunogenic recognition moiety, for example, a chemical hapten. Using a chemical hapten, preferably the HSG or DTPA hapten, high specificity of the linker for the antibody is exhibited. This occurs because antibodies raised to the HSG or DTPA hapten are known and the scFv portion of the antibody can be easily incorporated into the mutant bsAb. Thus, binding of the linker with the attached hapten would be highly specific for the scFv component.

The targetable construct may be monovalent or bivalent, with bivalent peptides being the preferred peptide. One exemplary targetable construct is IMP 192 (Ac-Lys(DTPA)-Tyr-Lys(DTPA)-Lys(TscG-Cys-)-NH₂). IMP 192 binds both Tc-99m and In-111 for diagnosis, and Re-188 and Re-186 for therapy. IMP 192 also binds bivalent DTPA-peptides with tyrosine.

In the methods of the invention, the targetable construct may comprise one or more radioactive isotopes useful for detecting diseased tissue. Particularly useful diagnostic radionuclides include, but are not limited to, ¹⁸F, ⁵²Fe, ⁶²Cu, ⁶⁴Cu, ⁶⁷Cu, ⁶⁷Ga, ⁶⁸Ga, ⁸⁶Y, ⁸⁹Zr, ^{94m}Tc, ⁹⁴Tc, ^{99m}Tc, ¹¹¹In, ¹²³I, ¹²⁴I, ¹²⁵I, ¹³¹I, ¹⁵⁴⁻¹⁵⁸Gd, ¹⁷⁷Lu, ³²P, ¹⁸⁸Re, ⁹⁰Y, or other gamma-, beta-, or positron-emitters, preferably with an energy in the range of 20 to 4,000 keV, more preferably in the range of 25 to 4,000 keV, and even more preferably in the range of 20 to 1,000 keV, and still more preferably in the range of 70 to 700 keV.

In the methods of the invention, the targetable construct may comprise one or more radioactive isotopes useful for treating diseased tissue. Particularly useful therapeutic radionuclides include, but are not limited to ³²P, ³³P, ⁴⁷Sc, ⁶⁴Cu, ⁶⁷Cu, ⁶⁷Ga, ⁹⁰Y, ¹¹¹Ag, ¹¹¹In, ¹²⁵I, ¹³¹I, ¹⁴²Pr, ¹⁵³Sm, ¹⁶¹Tb, ¹⁶⁶Dy, ¹⁶⁶Ho, ¹⁷⁷Lu, ¹⁸⁶Re, ¹⁸⁸Re, ¹⁸⁹Re, ²¹²Pb, ²¹²Bi, ²¹³Bi, ²¹¹At, ²²³Ra and ²²⁵Ac. The therapeutic radionuclide preferably has an energy in the range of 60 to 700 keV.

In the methods of the invention, the targetable construct may comprise one or more image enhancing agents for use in magnetic resonance imaging (MRI). By way of non-limiting example, the targetable compound comprises one or more paragmagnetic ions, such as Mn, Fe, and Gd.

In the methods of the invention, the targetable construct may comprise one or more image enhancing agents for use in ultrasound. By way of non-limiting example, the targetable construct comprises one or more ultrasound imaging agents. In one such embodiment, the targetable construct is a liposome with a bivalent DTPA-peptide covalently attached to the outside surface of the liposome lipid membrane. Optionally, said liposome may be gas filled.

IV. Chelate Moieties

The presence of hydrophilic chelate moieties on the linker moieties helps to ensure rapid in vivo clearance. In addition to hydrophilicity, chelators are chosen for their metal-binding properties, and are changed at will since, at least for those linkers whose bsAb epitope is part of the peptide or is a non-chelate chemical hapten, recognition of the metal-chelate complex is no longer an issue.

Particularly useful metal-chelate combinations include 2-benzyl-DTPA and its monomethyl and cyclohexyl analogs, used with ⁴⁷Sc, ⁵²Fe, ⁵⁵Co, ⁶⁷Ga, ⁶⁸Ga, ¹¹¹In, ⁸⁹Zr, 90Y, ¹⁶¹Tb, ¹⁷⁷Lu, ²¹²Bi, ²¹³Bi, and ²²⁵Ac for radio-imaging and RAIT. The same chelators, when complexed with non-radioactive metals, such as Mn, Fe and Gd can be used for MRI, when used along with the mutant bsAbs of the invention. Macrocyclic chelators such as NOTA (1,4,7-triaza-cyclononane-N,N′,N″-triacetic acid), DOTA, and TETA (p-bromoacetamido-benzyl-tetraethylaminetetraacetic acid) are of use with a variety of metals and radiometals, most particularly with radionuclides of Ga, Y and Cu, respectively.

DTPA and DOTA-type chelators, where the ligand includes hard base chelating functions such as carboxylate or amine groups, are most effective for chelating hard acid cations, especially Group Ia and Group IIIa metal cations. Such metal-chelate complexes can be made very stable by tailoring the ring size to the metal of interest. Other ring-type chelators such as macrocyclic polyethers are of interest for stably binding nuclides such as ²²³Ra for RAIT. Porphyrin chelators may be used with numerous radiometals, and are also useful as certain cold metal complexes for bsAb-directed immuno-phototherapy. More than one type of chelator may be conjugated to a carrier to bind multiple metal ions, e.g., cold ions, diagnostic radionuclides and/or therapeutic radionuclides. Particularly useful therapeutic radionuclides include, but are not limited to ³²P, ³³P, ⁴⁷Sc, ⁶⁴Cu, ⁶⁷Cu, ⁶⁷Ga, ⁹⁰Y, ¹¹¹Ag, ¹¹¹In, ¹²⁵I, ¹³¹I, ¹⁴²Pr, ¹⁵³Sm, ¹⁶¹Tb, ¹⁶⁶Dy, ¹⁶⁶Ho, ¹⁷⁷Lu, ¹⁸⁶ Re, ¹⁸⁸Re, ¹⁸⁹Re, ²¹²Pb, ²¹²Bi, ²¹³Bi, ²¹¹At, ²²³Ra and ²²⁵Ac. Particularly useful diagnostic radionuclides include, but are not limited to, ¹⁸F, ⁵²Fe, ⁶²Cu, ⁶⁴Cu ⁶⁷Cu, ⁶⁷Ga, ⁶⁸Ga, ⁸⁶Y, ⁸⁹Zr, ^{94m}Tc, ⁹⁴Tc, ^{99m}Tc, ¹¹¹In, ¹²³I, ¹²⁴I, ¹²⁵I, ¹³¹I, ¹⁵⁴⁻¹⁵⁸Gd and ¹⁷⁵Lu.

Chelators such as those disclosed in U.S. Pat. No. 5,753,206, especially thiosemi-carbazonylglyoxylcysteine (Tscg-Cys) and thiosemicarbazinyl-acetylcysteine (Tsca-Cys) chelators are advantageously used to bind soft acid cations of Tc, Re, Bi and other transition metals, lanthamides and actinides that are tightly bound to soft base ligands, especially sulfur- or phosphorus-containing ligands. It can be useful to link more than one type of chelator to a peptide, e.g., a DTPA or similar chelator for, say In(III) cations, and a thiol-containing chelator, e.g., Tscg-Cys, for Tc cations. Because antibodies to a di-DTPA hapten are known (Barbet '395, supra) and are readily coupled to a targeting antibody to form a bsAb, it is possible to use a peptide hapten with cold diDTPA chelator and another chelator for binding a radioisotope, in a pretargeting protocol, for targeting the radioisotope. One example of such a peptide is Ac-Lys(DTPA)-Tyr-Lys(DTPA)-Lys(Tscg-Cys-)-NH₂. This peptide can be preloaded with In(III) and then labeled with 99m-Tc cations, the In(III) ions being preferentially chelated by the DTPA and the Tc cations binding preferentially to the thiol-containing Tscg-Cys. Other hard acid chelators such as NOTA, DOTA, TETA and the like can be substituted for the DTPA groups, and Mabs specific to them can be produced using analogous techniques to those used to generate the anti-di-DTPA Mab.

It will be appreciated that two different hard acid or soft acid chelators can be incorporated into the linker, e.g., with different chelate ring sizes, to bind preferentially to two different hard acid or soft acid cations, due to the differing sizes of the cations, the geometries of the chelate rings and the preferred complex ion structures of the cations. This will permit two different metals, one or both of which may be radioactive or useful for MRI enhancement, to be incorporated into a linker for eventual capture by a pretargeted bsAb.

Preferred chelators include NOTA, DOTA and Tscg and combinations thereof. These chelators have been incorporated into a chelator-peptide conjugate motif as exemplified in the following constructs:

| | |
|---|---|
| (a) | DOTA-Phe-Lys(HSG)-D-Tyr-Lys(HSG)-NH₂; |
| (b) | DOTA-Phe-Lys(HSG)-Tyr-Lys(HSG)-NH₂; |
| (c) | Ac-Lys(HSG)D-Tyr-Lys(HSG)-Lys(Tscg-Cys)-NH₂; |
| (d) | 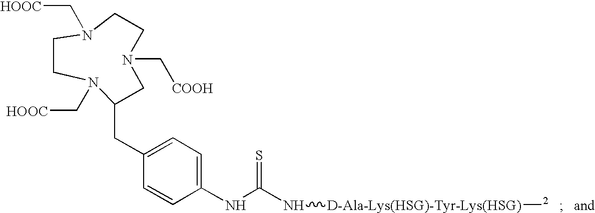 |
| (e) | 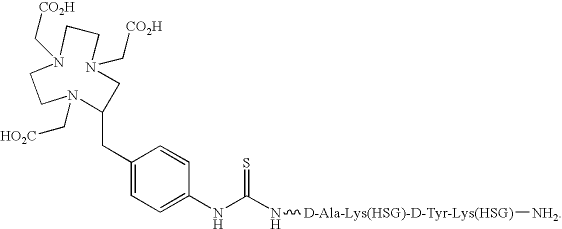 |

The chelator-peptide conjugates (d) and (e), above, has been shown to bind ⁶⁸Ga and is thus useful in positron emission tomography (PET) applications.

Chelators are coupled to the linker moieties using standard chemistries which are discussed more fully in the working Examples below. Briefly, the synthesis of the peptide Ac-Lys(HSG)D-Tyr-Lys(HSG)-Lys(Tscg-Cys-)-NH₂ was accomplished by first attaching Aloc-Lys(Fmoc)-OH to a Rink amide resin on the peptide synthesizer. The protecting group abbreviations “Aloc” and “Fmoc” used herein refer to the groups allyloxycarbonyl and fluorenylmethyloxy carbonyl. The Fmoc-Cys(Trt)-OH and TscG were then added to the side chain of the lysine using standard Fmoc automated synthesis protocols to form the following peptide: Aloc-Lys(Tscg-Cys(Trt)-rink resin. The Aloc group was then removed. The peptide synthesis was then continued on the synthesizer to make the following peptide: (Lys(Aloc)-D-Tyr-Lys(Aloc)-Lys(Tscg-Cys(Trt)-)-rink resin. Following N-terminus acylation, and removal of the side chain Aloc protecting groups. The resulting peptide was then treated with activated N-trityl-HSG-OH until the resin gave a negative test for amines using the Kaiser test. See Karacay et al. Bioconjugate Chem. 11:842-854 (2000). The synthesis of Ac-Lys(HSG)D-Tyr-Lys(HSG)-Lys(Tscg-Cys-)-NH₂, as well as the syntheses of DOTA-Phe-Lys(HSG)-D-Tyr-Lys(HSG)-NH₂; and DOTA-Phe-Lys(HSG)-Tyr-Lys(HSG)-NH₂ are described in greater detail below.

V. General Methods for Preparation of Metal Chelates

Chelator-peptide conjugates may be stored for long periods as solids. They may be metered into unit doses for metal-binding reactions, and stored as unit doses either as solids, aqueous or semi-aqueous solutions, frozen solutions or lyophilized preparations. They may be labeled by well-known procedures. Typically, a hard acid cation is introduced as a solution of a convenient salt, and is taken up by the hard acid chelator and possibly by the soft acid chelator. However, later addition of soft acid cations leads to binding thereof by the soft acid chelator, displacing any hard acid cations which may be chelated therein. For example, even in the presence of an excess of cold ¹¹¹InCl₃, labeling with 99m-Tc(V) glucoheptonate or with Tc cations generated in situ with stannous chloride and Na99m-TcO₄ proceeds quantitatively on the soft acid chelator. Other soft acid cations such as ¹⁸⁶Re, ¹⁸⁸Re, ²¹³Bi and divalent or trivalent cations of Mn, Co, Ni, Pb, Cu, Cd, Au, Fe, Ag (monovalent), Zn and Hg, especially ⁶⁴Cu and ⁶⁷Cu, and the like, some of which are useful for radioimmunodiagnosis or radioimmunotherapy, can be loaded onto the linker peptide by analogous methods. Re cations also can be generated in situ from perrhenate and stannous ions or a prereduced rhenium glucoheptonate or other transchelator can be used. Because reduction of perrhenate requires more stannous ion (typically above 200 μg/mL final concentration) than is needed for the reduction of Tc, extra care needs to be taken to ensure that the higher levels of stannous ion do not reduce sensitive disulfide bonds such as those present in disulfide-cyclized peptides. During radiolabeling with rhenium, similar procedures are used as are used with the Tc-99m. A preferred method for the preparation of ReO metal complexes of the Tscg-Cys-ligands is by reacting the peptide with ReOCl₃(P(Ph₃)₂ but it is also possible to use other reduced species such as ReO(ethylenediamine)₂.

VI. Methods for Raising Antibodies

Antibodies to peptide backbones are generated by well-known methods for Ab production. For example, injection of an immunogen, such as (peptide)ₙ-KLH, wherein KLH is keyhole limpet hemocyanin, and n=1-30, in complete Freund's adjuvant, followed by two subsequent injections of the same immunogen suspended in incomplete Freund's adjuvant into immunocompetent animals, is followed three days after an i.v. boost of antigen, by spleen cell harvesting. Harvested spleen cells are then fused with Sp2/0-Ag14 myeloma cells and culture supernatants of the resulting clones analyzed for anti-peptide reactivity using a direct-binding ELISA. Fine specificity of generated Abs can be analyzed for by using peptide fragments of the original immunogen. These fragments can be prepared readily using an automated peptide synthesizer. For Ab production, enzyme-deficient hybridomas are isolated to enable selection of fused cell lines. This technique also can be used to raise antibodies to one or more of the chelates comprising the linker, e.g., in(III)-DTPA chelates. Monoclonal mouse antibodies to an In(III)-di-DTPA are known (Barbet '395 supra).

The mutant bispecific antibodies used in the present invention are specific to a variety of cell surface or intracellular tumor-associated antigens as marker substances. These markers may be substances produced by the tumor or may be substances which accumulate at a tumor site, on tumor cell surfaces or within tumor cells, whether in the cytoplasm, the nucleus or in various organelles or sub-cellular structures. Among such tumor-associated markers are those disclosed by Herberman, “Immunodiagnosis of Cancer”, in Fleisher ed., “The Clinical Biochemistry of Cancer”, page 347 (American Association of Clinical Chemists, 1979) and in U.S. Pat. Nos. 4,150,149; 4,361,544; and 4,444,744.

Tumor-associated markers have been categorized by Herberman, supra, in a number of categories including oncofetal antigens, placental antigens, oncogenic or tumor virus associated antigens, tissue associated antigens, organ associated antigens, ectopic hormones and normal antigens or variants thereof. Occasionally, a sub-unit of a tumor-associated marker is advantageously used to raise antibodies having higher tumor-specificity, e.g., the beta-subunit of human chorionic gonadotropin (HCG) or the gamma region of carcino embryonic antigen (CEA), which stimulate the production of antibodies having a greatly reduced cross-reactivity to non-tumor substances as disclosed in U.S. Pat. Nos. 4,361,644 and 4,444,744.

Another marker of interest is transmembrane activator and CAML-interactor (TACI). See Yu et al. Nat. Immunol. 1:252-256 (2000). Briefly, TACI is a marker for B-cell malignancies (e.g., lymphoma). Further it is known that TACI and B cell maturation antigen (BCMA) are bound by the tumor necrosis factor homolog a proliferation-inducing ligand (APRIL). APRIL stimulates in vitro proliferation of primary B and T cells and increases spleen weight due to accumulation of B cells in vivo. APRIL also competes with TALL-I (also called BLyS or BAFF) for receptor binding. Soluble BCMA and TACI specifically prevent binding of APRIL and block APRIL-stimulated proliferation of primary B cells. BCMA-Fc also inhibits production of antibodies against keyhole limpet hemocyanin and Pneumovax in mice, indicating that APRIL and/or TALL-I signaling via BCMA and/or TACI are required for generation of humoral immunity. Thus, APRIL-TALL-I and BCMA-TACI form a two ligand-two receptor pathway involved in stimulation of B and T cell function.

After the initial raising of antibodies to the immunogen, the antibodies can be sequenced and subsequently prepared by recombinant techniques. Humanization and chimerization of murine antibodies and antibody fragments are well known to those skilled in the art. For example, humanized monoclonal antibodies are produced by transferring mouse complementary determining regions from heavy and light variable chains of the mouse immunoglobulin into a human variable domain, and then, substituting human residues in the framework regions of the murine counterparts. The use of antibody components derived from humanized monoclonal antibodies obviates potential problems associated with the immunogenicity of murine constant regions. General techniques for cloning murine immunoglobulin variable domains are described, for example, by the publication of Orlandi et al., Proc. Nat'l Acad. Sci. USA 86: 3833 (1989), which is incorporated by reference in its entirety. Techniques for producing humanized Mabs are described, for example, by Jones et al., Nature 321: 522 (1986), Riechmann et al., Nature 332: 323 (1988), Verhoeyen et al., Science 239: 1534 (1988), Carter et al., Proc. Nat'l Acad. Sci. USA 89: 4285 (1992), Sandhu, Crit. Rev. Biotech. 12: 437 (1992), and Singer et al., J. Immun. 150: 2844 (1993), each of which is hereby incorporated in its entirety by reference.

Alternatively, fully human antibodies can be obtained from transgenic non-human animals. See, e.g., Mendez et al., Nature Genetics, 15: 146-156 (1997); U.S. Pat. No. 5,633,425. For example, human antibodies can be recovered from transgenic mice possessing human immunoglobulin loci. The mouse humoral immune system is humanized by inactivating the endogenous immunoglobulin genes and introducing human immunoglobulin loci. The human immunoglobulin loci are exceedingly complex and comprise a large number of discrete segments which together occupy almost 0.2% of the human genome. To ensure that transgenic mice are capable of producing adequate repertoires of antibodies, large portions of human heavy- and light-chain loci must be introduced into the mouse genome. This is accomplished in a stepwise process beginning with the formation of yeast artificial chromosomes (YACs) containing either human heavy- or light-chain immunoglobulin loci in germline configuration. Since each insert is approximately 1 Mb in size, YAC construction requires homologous recombination of overlapping fragments of the immunoglobulin loci. The two YACs, one containing the heavy-chain loci and one containing the light-chain loci, are introduced separately into mice via fusion of YAC-containing yeast spheroblasts with mouse embryonic stem cells. Embryonic stem cell clones are then microinjected into mouse blastocysts. Resulting chimeric males are screened for their ability to transmit the YAC through their germline and are bred with mice deficient in murine antibody production. Breeding the two transgenic strains, one containing the human heavy-chain loci and the other containing the human light-chain loci, creates progeny which produce human antibodies in response to immunization.

Unrearranged human immunoglobulin genes also can be introduced into mouse embryonic stem cells via microcell-mediated chromosome transfer (MMCT). See, e.g., Tomizuka et al., Nature Genetics, 16: 133 (1997). In this methodology microcells containing human chromosomes are fused with mouse embryonic stem cells. Transferred chromosomes are stably retained, and adult chimeras exhibit proper tissue-specific expression.

As an alternative, an antibody or antibody fragment of the present invention may be derived from human antibody fragments isolated from a combinatorial immunoglobulin library. See, e.g., Barbas et al., METHODS: A Companion to Methods in Enzymology 2: 119 (1991), and Winter et al., Ann. Rev. Immunol. 12: 433 (1994), which are incorporated in their entirety by reference. Many of the difficulties associated with generating monoclonal antibodies by B-cell immortalization can be overcome by engineering and expressing antibody fragments in E. coli, using phage display. To ensure the recovery of high affinity, monoclonal antibodies a combinatorial immunoglobulin library must contain a large repertoire size. A typical strategy utilizes mRNA obtained from lymphocytes or spleen cells of immunized mice to synthesize cDNA using reverse transcriptase. The heavy- and light-chain genes are amplified separately by PCR and ligated into phage cloning vectors. Two different libraries are produced, one containing the heavy-chain genes and one containing the light-chain genes. Phage DNA is islolated from each library, and the heavy- and light-chain sequences are ligated together and packaged to form a combinatorial library. Each phage contains a random pair of heavy- and light-chain cDNAs and upon infection of E. coli directs the expression of the antibody chains in infected cells. To identify an antibody that recognizes the antigen of interest, the phage library is plated, and the antibody molecules present in the plaques are transferred to filters. The filters are incubated with radioactively labeled antigen and then washed to remove excess unbound ligand. A radioactive spot on the autoradiogram identifies a plaque that contains an antibody that binds the antigen. Cloning and expression vectors that are useful for producing a human immunoglobulin phage library can be obtained, for example, from STRATAGENE Cloning Systems (La Jolla, Calif.).

A similar strategy can be employed to obtain high-affinity scFv. See, e.g., Vaughn et al., Nat. Biotechnol., 14: 309-314 (1996). An scFv library with a large repertoire can be constructed by isolating V-genes from non-immunized human donors using PCR primers corresponding to all known V_{H}, V_{κ} and V_{λ} gene families. Following amplification, the V_{κ} and V_{λ} pools are combined to form one pool. These fragments are ligated into a phagemid vector. The scFv linker, (Gly₄, Ser)₃, is then ligated into the phagemid upstream of the V_{L} fragment. The V_{H} and linker-V_{L} fragments are amplified and assembled on the J_{H} region. The resulting V_{H}-linker-V_{L} fragments are ligated into a phagemid vector. The phagemid library can be panned using filters, as described above, or using immunotubes (Nunc; Maxisorp). Similar results can be achieved by constructing a combinatorial immunoglobulin library from lymphocytes or spleen cells of immunized rabbits and by expressing the scFv constructs in P. pastoris. See, e.g., Ridder et al., Biotechnology, 13: 255-260 (1995). Additionally, following isolation of an appropriate scFv, antibody fragments with higher binding affinities and slower dissociation rates can be obtained through affinity maturation processes such as CDR3 mutagenesis and chain shuffling. See, e.g., Jackson et al., Br. J. Cancer, 78: 181-188 (1998); Osbourn et al., Immunotechnology, 2: 181-196 (1996).

A variety of recombinant methods can be used to produce bi-specific antibodies and antibody fragments. For example, bi-specific antibodies and antibody fragments can be produced in the milk of transgenic livestock. See, e.g., Colman, A., Biochem. Soc. Symp., 63: 141-147, 1998; U.S. Pat. No. 5,827,690. Two DNA constructs are prepared which contain, respectively, DNA segments encoding paired immunoglobulin heavy and light chains. The fragments are cloned into expression vectors which contain a promoter sequence that is preferentially expressed in mammary epithelial cells. Examples include, but are not limited to, promoters from rabbit, cow and sheep casein genes, the cow α-lactoglobulin gene, the sheep β-lactoglobulin gene and the mouse whey acid protein gene. Preferably, the inserted fragment is flanked on its 3′ side by cognate genomic sequences from a mammary-specific gene. This provides a polyadenylation site and transcript-stabilizing sequences. The expression cassettes are coinjected into the pronuclei of fertilized, mammalian eggs, which are then implanted into the uterus of a recipient female and allowed to gestate. After birth, the progeny are screened for the presence of both transgenes by Southern analysis. In order for the antibody to be present, both heavy and light chain genes must be expressed concurrently in the same cell. Milk from transgenic females is analyzed for the presence and functionality of the antibody or antibody fragment using standard immunological methods known in the art. The antibody can be purified from the milk using standard methods known in the art.

A chimeric Ab is constructed by ligating the cDNA fragment encoding the mouse light variable and heavy variable domains to fragment encoding the C domains from a human antibody. Because the C domains do not contribute to antigen binding, the chimeric antibody will retain the same antigen specificity as the original mouse Ab but will be closer to human antibodies in sequence. Chimeric Abs still contain some mouse sequences, however, and may still be immunogenic. A humanized Ab contains only those mouse amino acids necessary to recognize the antigen. This product is constructed by building into a human antibody framework the amino acids from mouse complementarity determining regions.

VII. General Methods for Design and Expression of Mutant Bi-Specific Antibodies

Various mutagenesis techniques may be used to construct the mutant bsAb of the present invention. A person of ordinary skill in the art is well acquainted with such techniques. For example, an expression vector for the mutant bsAb may be obtained by constructing a mutated HC fragment, subcloning this fragment into the expression vector for the parent bsAb to replace the corresponding wild type fragment, and transfecting a host cell with the vector.

In order to obtain an expression vector for the parent bsAb, a person of ordinary skill in the art can use techniques readily available. Some of these techniques are disclosed in U.S. application Ser. No. 09/337,756 filed Jun. 22, 1999, the entire contents of which are incorporated by reference. Briefly, in order to construct an expression vector of a parent bsAb, such as hMN14IgG-(734 scFv)₂, the gene segment encoding a single chain 734 Fv (734scFv) may be constructed. The 734scFv segment may be linked to the 3′-end of human gamma-chain gene through a DNA fragment coding for a short flexible linker (sL) (Coloma & Morrison 1997 p.787/id) resulting in a fusion gene sequence for C_{H}1-Hinge-C_{H}2-C_{H}3-sL-734scFv (C_{H}-scFv). The CH-scFv fusion gene segment can then be linked to the sequence for hMN-14 V_{H} in an expression vector, hMN14pdHL2, which also contained hMN-14 light chain gene segment, as well as a dhfr gene for selection of transfectants and subsequent amplification of the transfected sequences (Dorai & Moore 1987 p. 815/id and Gillies, Lo et al. 1989 p. 131/id). The vector encoding hMN14IgG-(734scFV)₂ (bsAb2pdHL2) may be transfected into Sp2/0 myeloma cells for expression of the fusion bsAb. The bsAb, hMN14IgG-(734scFv)₂, can be purified from culture supernatants by affinity chromatography and analyzed by SDS-PAGE. To evaluate the immunoreactivities of the different biding moieties within a parent or mutant bsAb, competitive ELISA binding assays may be performed.

A bsAbs of IgG-scFv with other specificities and the respective mutant bsAbs can be generated by substitution of only the variable region sequences of the IgG and/or the scFv with those of other Abs. The CDR grafted mutant bsAb can be generated by substitution of only the variable region sequences of the IgG or scFv with those of the CDR grafted Abs. Typically, this “CDR-grafting” technology has been applied to the generation of recombinant, pharmaceutical antibodies consisting of murine CDRs, human variable region frameworks and human constant regions (eg Riechmann, L. et al, (1988) Nature, 332, 323-327). Such “reshaped” or “humanized” antibodies have less murine content than chimeric antibodies and retain the human constant regions necessary for the stimulation of human Fe dependent effector functions. In consequence, CDR grafted antibodies are less likely than chimeric antibodies to evoke a HAMA response when administered to humans, their half-life in circulation should approach that of natural human antibodies and their diagnostic and therapeutic value is enhanced.

In practice, for the generation of efficacious humanized antibodies retaining the specificity of the original murine antibody, it is not usually sufficient simply to substitute CDRs. In addition there is a requirement for the inclusion of a small number of critical murine antibody residues in the human variable region. The identity of these residues depends on the structure of both the original murine antibody and the acceptor human antibody. British Patent Application Number 9019812.8 (the entire contents of which is incorporated by reference) discloses a method for identifying a minimal number of substitutions of foreign residues sufficient to promote efficacious antigen binding. In one embodiment of the present invention, the Fvs and scFvs of the mutant fusion protein are CDR-grafted murine Fvs and scFvs. In another embodiment of the present invention, the Fvs and scFvs of the mutant fusion protein are humanized. In one embodiment, the Fvs are derived from and the scFvs are 734scFv. In a preferred embodiment of the present invention, the mutant fusion protein is hMN-14IgG^{I253A}-(734scFV)₂.

VIII. Methods of Administration Mutant bsAbs

The present invention contemplates the use of the inventive bispecific antibodies and targetable constructs in treating and/or imaging normal tissue and organs using the methods described in U.S. Pat. Nos. 6,126,916; 6,077,499; 6,010,680; 5,776,095; 5,776,094; 5,776,093; 5,772,981; 5,753,206; 5,746,996; 5,697,902; 5,328,679; 5,128,119; 5,101,827; and 4,735,210. Additional methods are described in U.S. application Ser. No. 09/337,756 filed Jun. 22, 1999 and in U.S. application Ser. No. 09/823,746, filed Apr. 3, 2001. As used herein, the term “tissue” refers to tissues, including but not limited to, tissues from the ovary, thymus, parathyroid or spleen. Exemplary diseases and conditions that can be treated with the mutant bsAb of the present invention are immune dysregulation disease, an autoimmune disease, organ graft rejection or graft vs. host disease. Immunothereapy of autoimmune disorders using antibodies which target B-cells is described in WO 00/74718 m which claims priority to U.S. Provisional Application No. 60/138,284, the contents of which is incorporated herein in its entirety. Exemplary autoimmune diseases are acute idiopathic thrombocytopenic purpura, chronic idiopathic thrombocytopenic purpura, dermatomyositis, Sydenham's chorea, myasthenia gravis, systemic lupus erythematosus, lupus nephritis, rheumatic fever, polyglandular syndromes, bullous pemphigoid, diabetes mellitus, Henoch-Schonlein purpura, post-streptococcalnephritis, erythema nodosurn, Takayasu's arteritis, Addison's disease, rheumatoid arthritis, multiple sclerosis, sarcoidosis, ulcerative colitis, erythema multiforme, IgA nephropathy, polyarteritis nodosa, ankylosing spondylitis, Goodpasture's syndrome, thromboangitisubiterans, Sjogren's syndrome, primary biliary cirrhosis, Hashimoto's thyroiditis, thyrotoxicosis, scleroderma, chronic active hepatitis, polymyositis/dermatomyositis, polychondritis, parnphigus vulgaris, Wegener's granulomatosis, membranous nephropathy, amyotrophic lateral sclerosis, tabes dorsalis, giant cell arteritis/polymyalgia, perniciousanemia, rapidly progressive glomerulonephritis and fibrosing alveolitis.

The mutant bsAb of the present invention may be used in a pretargeting method as the primary targeting species. In pretargeting methods, the mutant bsAb is administered. Once sufficient accretion of the primary targeting species is achieved, a targetable construct is administered. The targetable construct comprises a binding site which recognizes the available binding site of the primary targeting species and a diagnostic or therapeutic agent. Exemplary targetable constructs are described above. The doses and timing of the reagents can be readily worked out by a skilled artisan, and are dependent on the specific nature of the reagents employed. A pretargeting method may be performed with or without the use of a clearing agent.

After sufficient time has passed for the bsAb to target to the diseased tissue, the diagnostic agent is administered. Subsequent to administration of the diagnostic agent, imaging can be performed. Tumors can be detected in body cavities by means of directly or indirectly viewing various structures to which light of the appropriate wavelength is delivered and then collected. Lesions at any body site can be viewed so long as nonionizing radiation can be delivered and recaptured from these structures. For example, PET which is a high resolution, non-invasive, imaging technique can be used with the inventive antibodies for the visualization of human disease. In PET, 511 keV gamma photons produced during positron annihilation decay are detected when using F-18 as the positron-emitter.

The invention generally contemplates the use of diagnostic agents which emit 25-600 keV gamma particles and/or positrons. Examples of such agents include, but are not limited to ¹⁸F, ⁵²Fe, ⁶²Cu, ⁶⁴Cu, ⁶⁷Cu, ⁶⁷Ga, ⁶⁸Ga, ⁸⁶Y, ⁸⁹Zr, ^{94m}Tc, 94Tc, ^{99m}Tc, ¹¹¹In, ¹²³I, ¹²⁴I, ¹²⁵I, ¹³¹I, ¹⁵⁴⁻¹⁵⁸Gd and ¹⁷⁵Lu.

Detection with intraoperative/endoscopic probes is also contemplated in methods involving a mutant bsAb of the present invention and a targetable construct which is a peptide labeled with I-125. Such methods are disclosed in U.S. Pat. Nos. 5,716,595 and 6,096,289, the entire contents of which are incorporated by reference.

The present mutant bsAb can be used in a method of photodynamic therapy (PDT) as discussed in U.S. Pat. Nos. 6,096,289; 4,331,647; 4,818,709; 4,348,376; 4,361,544; 4,444,744; 5,851,527.

In PDT, a photosensitizer, e.g., a hematoporphyrin derivative such as dihematoporphyrin ether, is administered to a subject. Anti-tumor activity is initiated by the use of light, e.g., 630 nm. Alternate photosensitizers can be utilized, including those useful at longer wavelengths, where skin is less photosensitized by the sun. Examples of such photosensitizers include, but are not limited to, benzoporphyrin monoacid ring A (BPD-MA), tin etiopurpurin (SnET2), sulfonated aluminum phthalocyanine (AlSPc) and lutetium texaphyrin (Lutex).

Additionally, in PDT, a diagnostic agent is injected, for example, systemically, and laser-induced fluorescence can be used by endoscopes to detect sites of cancer which have accreted the light-activated agent. For example, this has been applied to fluorescence bronchoscopic disclosure of early lung tumors. Doiron et al. Chest 76:32 (1979). In another example, the antibodies and antibody fragments can be used in single photon emission. For example, a Tc-99m-labeled diagnostic agent can be administered to a subject following administration of the inventive antibodies or antibody fragments. The subject is then scanned with a gamma camera which produces single-photon emission computed tomographic images and defines the lesion or tumor site.

Therapeutically useful immunoconjugates can be obtained by conjugating photoactive agents or dyes to an antibody composite. Fluorescent and other chromogens, or dyes, such as porphyrins sensitive to visible light, have been used to detect and to treat lesions by directing the suitable light to the lesion. In therapy, this has been termed photoradiation, phototherapy, or photodynamic therapy (Jori et al. (eds.), Photodynamic Therapy of Tumors and Other Diseases (Libreria Progetto 1985); van den Bergh, Chem. Britain 22:430 (1986)). Moreover, monoclonal antibodies have been coupled with photoactivated dyes for achieving phototherapy. Mew et al., J. Immunol. 130:1473 (1983); idem., Cancer Res. 45:4380 (1985); Oseroff et al., Proc. Natl. Acad. Sci. USA 83:8744 (1986); idem., Photochem. Photobiol. 46:83 (1987); Hasan et al., Prog. Clin. Biol. Res. 288:471 (1989); Tatsuta et al., Lasers Surg. Med. 9:422 (1989); Pelegrin et al., Cancer 67:2529 (1991). However, these earlier studies did not include use of endoscopic therapy applications, especially with the use of antibody fragments or subfragments. Thus, the present invention contemplates the therapeutic use of immunoconjugates comprising photoactive agents or dyes.

The linker moiety may also be conjugated to an enzyme capable of activating a prodrug at the target site or improving the efficacy of a normal therapeutic by controlling the body's detoxification pathways. Following administration of the bsAb, an enzyme conjugated to the linker moiety, a low MW hapten recognized by the second arm of the bsAb (the scFv component), is administered. After the enzyme is pretargeted to the target site, a cytotoxic drug is injected, which is known to act at the target site. The drug may be one which is detoxified by the mammal's ordinary detoxification processes. For example, the drug may be converted into the potentially less toxic glucuronide in the liver. The detoxified intermediate can then be reconverted to its more toxic form by the pretargeted enzyme at the target site. Alternatively, an administered prodrug can be converted to an active drug by the pretargeted enzyme. The pretargeted enzyme improves the efficacy of the treatment by recycling the detoxified drug. This approach can be adopted for use with any enzyme-drug pair.

Certain cytotoxic drugs that are useful for anticancer therapy are relatively insoluble in serum. Some are also quite toxic in an unconjugated form, and their toxicity is considerably reduced by conversion to prodrugs. Conversion of a poorly soluble drug to a more soluble conjugate, e.g., a glucuronide, an ester of a hydrophilic acid or an amide of a hydrophilic amine, will improve its solubility in the aqueous phase of serum and its ability to pass through venous, arterial or capillary cell walls and to reach the interstitial fluid bathing the tumor. Cleavage of the prodrug deposits the less soluble drug at the target site. Many examples of such prodrug-to-drug conversions are disclosed in Hansen U.S. Pat. No. 5,851,527.

Conversion of certain toxic substances such as aromatic or alicyclic alcohols, thiols, phenols and amines to glucuronides in the liver is the body's method of detoxifying them and making them more easily excreted in the urine. One type of antitumor drug that can be converted to such a substrate is epirubicin, a 4-epimer of doxorubicin (Adriamycin), which is an anthracycline glycoside and has been shown to be a substrate for human beta-D-glucuronidase See, e.g., Arcamone Cancer Res. 45:5995 (1985). Other analogues with fewer polar groups are expected to be more lipophilic and show greater promise for such an approach. Other drugs or toxins with aromatic or alicyclic alcohol, thiol or amine groups are candidates for such conjugate formation. These drugs, or other prodrug forms thereof, are suitable candidates for the site-specific enhancement methods of the present invention.

The prodrug CPT-11 (irinotecan) is converted in vivo by carboxylesterase to the active metabolite SN-38. One application of the invention, therefore, is to use a bsAb targeted against a tumor and a hapten (e.g. di-DTPA) followed by injection of a di-DTPA-carboxylesterase conjugate. Once a suitable tumor-to-background localization ratio has been achieved, the CPT-11 is given and the tumor-localized carboxylesterase serves to convert CPT-11 to SN-38 at the tumor. Due to its poor solubility, the active SN-38 will remain in the vicinity of the tumor and, consequently, will exert an effect on adjacent tumor cells that are negative for the antigen being targeted. This is a further advantage of the method. Modified forms of carboxylesterases have been described and are within the scope of the invention. See, e.g., Potter et al., Cancer Res. 58:2646-2651 (1998) and Potter et al., Cancer Res. 58:3627-3632 (1998).

Etoposide is a widely used cancer drug that is detoxified to a major extent by formation of its glucuronide and is within the scope of the invention. See, e.g., Hande et al. Cancer Res. 48:1829-1834 (1988). Glucuronide conjugates can be prepared from cytotoxic drugs and can be injected as therapeutics for tumors pre-targeted with mAb-glucuronidase conjugates. See, e.g., Wang et al. Cancer Res. 52:4484-4491 (1992). Accordingly, such conjugates also can be used with the pre-targeting approach described here. Similarly, designed prodrugs based on derivatives of daunomycin and doxorubicin have been described for use with carboxylesterases and glucuronidases. See, e.g., Bakina et al. J. Med Chem. 40:4013-4018 (1997). Other examples of prodrug/enzyme pairs that can be used within the present invention include, but are not limited to, glucuronide prodrugs of hydroxy derivatives of phenol mustards and beta-glucuronidase; phenol mustards or CPT-11 and carboxypeptidase; methotrexate-substituted alpha-amino acids and carboxypeptidase A; penicillin or cephalosporin conjugates of drugs such as 6-mercaptopurine and doxorubicin and beta-lactamase; etoposide phosphate and alkaline phosphatase.

The enzyme capable of activating a prodrug at the target site or improving the efficacy of a normal therapeutic by controlling the body's detoxification pathways may alternatively be conjugated to the hapten. The enzyme-hapten conjugate is administered to the subject following administration of the pre-targeting bsAb and is directed to the target site. After the enzyme is localized at the target site, a cytotoxic drug is injected, which is known to act at the target site, or a prodrug form thereof which is converted to the drug in situ by the pretargeted enzyme. As discussed above, the drug is one which is detoxified to form an intermediate of lower toxicity, most commonly a glucuronide, using the mammal's ordinary detoxification processes. The detoxified intermediate, e.g., the glucuronide, is reconverted to its more toxic form by the pretargeted enzyme and thus has enhanced cytotoxicity at the target site. This results in a recycling of the drug. Similarly, an administered prodrug can be converted to an active drug through normal biological processess. The pretargeted enzyme improves the efficacy of the treatment by recycling the detoxified drug. This approach can be adopted for use with any enzyme-drug pair.

The invention further contemplates the use of the inventive bsAb and the diagnostic agent(s) in the context of Boron Neutron Capture Therapy (BNCT) protocols. BNCT is a binary system designed to deliver ionizing radiation to tumor cells by neutron irradiation of tumor-localized ¹⁰B atoms. BNCT is based on the nuclear reaction which occurs when a stable isotope, isotopically enriched ¹⁰B (present in 19.8% natural abundance), is irradiated with thermal neutrons to produce an alpha particle and a ⁷Li nucleus. These particles have a path length of about one cell diameter, resulting in high linear energy transfer. Just a few of the short-range 1.7 MeV alpha particles produced in this nuclear reaction are sufficient to target the cell nucleus and destroy it. Success with BNCT of cancer requires methods for localizing a high concentration of ¹⁰B at tumor sites, while leaving non-target organs essentially boron-free. Compositions and methods for treating tumors in subjects using pre-targeting bsAb for BNCT are described in co-pending patent application Ser. No. 09/205,243 and can easily be modified for the purposes of the present invention.

It should also be noted that scFv component of the mutant bsAb of the present invention may also be specific to an enzyme.

A clearing agent may be used which is given between doses of the mutant bsAb and the targetable construct. The present inventors have discovered that a clearing agent of novel mechanistic action may be used with the invention, namely a glycosylated anti-idiotypic Fab′ fragment targeted against the disease targeting arm(s) of the bsAb. Anti-CEA (MN 14 Ab) x anti-peptide bsAb is given and allowed to accrete in disease targets to its maximum extent. To clear residual bsAb, an anti-idiotypic Ab to MN-14, termed WI2, is given, preferably as a glycosylated Fab′ fragment. The clearing agent binds to the bsAb in a monovalent manner, while its appended glycosyl residues direct the entire complex to the liver, where rapid metabolism takes place. Then the therapeutic which is associated with the linker moiety is given to the subject. The WI2 Ab to the MN-14 arm of the bsAb has a high affinity and the clearance mechanism differs from other disclosed mechanisms (see Goodwin et al., ibid), as it does not involve cross-linking, because the WI2-Fab′ is a monovalent moiety.

The present mutant bsAb can also be used in a method of ultrasound imaging. An ultrasound enhancement agent, such as a contrast agent, may be attached to a targetable construct, such as a bivalent DTPA peptide. By way of non-limiting example, an enhancement agent such as a liposome, preferably a gas-filled liposome may be used. In this method, the mutant bsAb would be administered first, followed by administration of the liposome-targetable construct complex. See Maresca, G. et al., Eur J. Radiol. Suppl. 2 S171-178 (1998); Demos, Sm. Et al. J. Drug Target 5 507-518 (1998); and Unger, E. et al., Am J. Cardiol. 81 58G-61G (1998).

The mutant bispecific antibody may be administered as one component of a multi-component treatment regimen. The mutant bispecific antibody may be administered before, during or after the administration of at least one therapeutic agent used to treat a disease or condition.

The use of an exemplary mutant bsAb in a pretargeting method, compared to the use of a parent bsAb in a pretargeting method is illustrated in Example 2. The data illustrates the accelerated rate of clearance of a mutant bsAb of the present invention as compared to the parent bsAb. Additionally, the data illustrates that a much larger amount of targetable construct is trapped in the blood when the parent bsAb is used as compared to when the mutant bsAb is used.

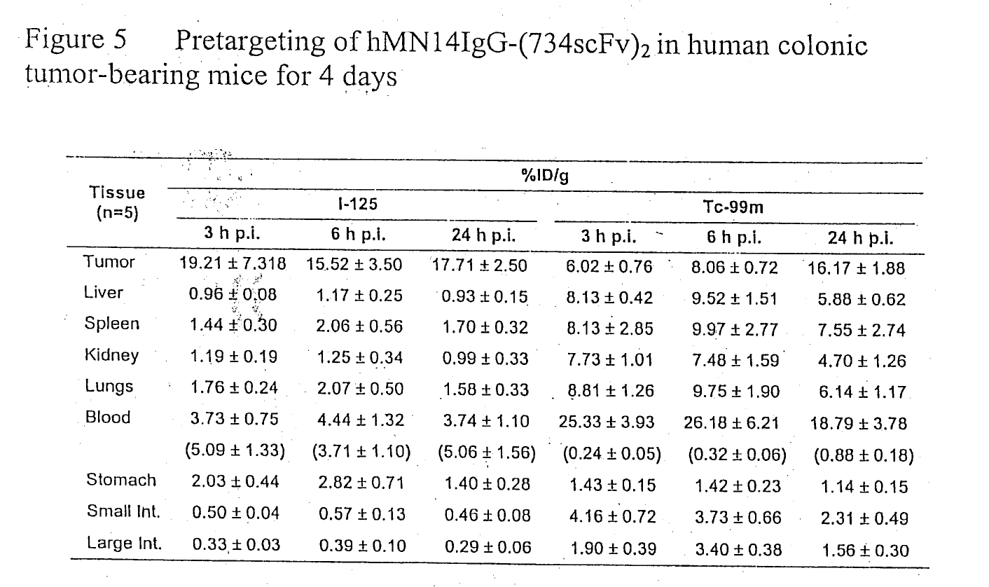

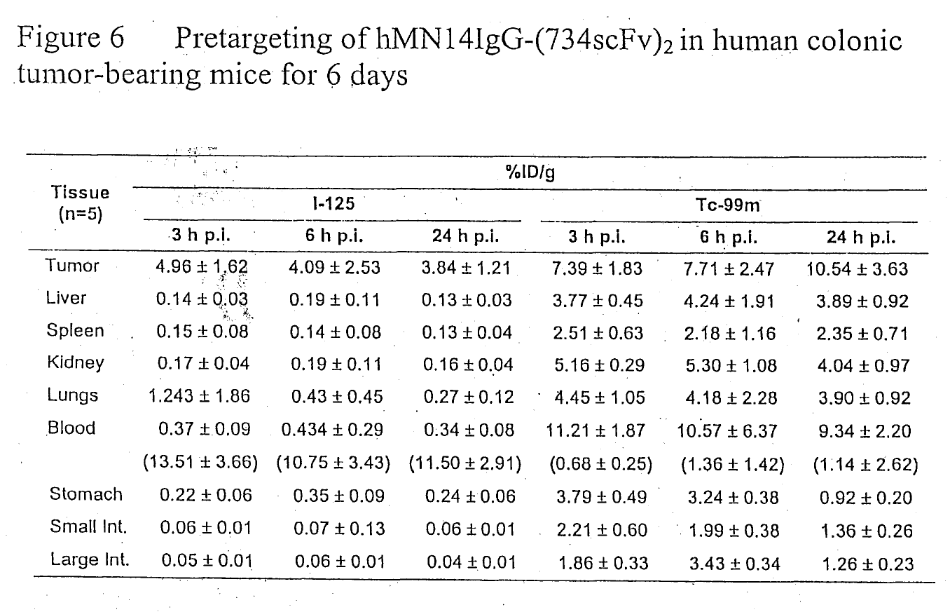

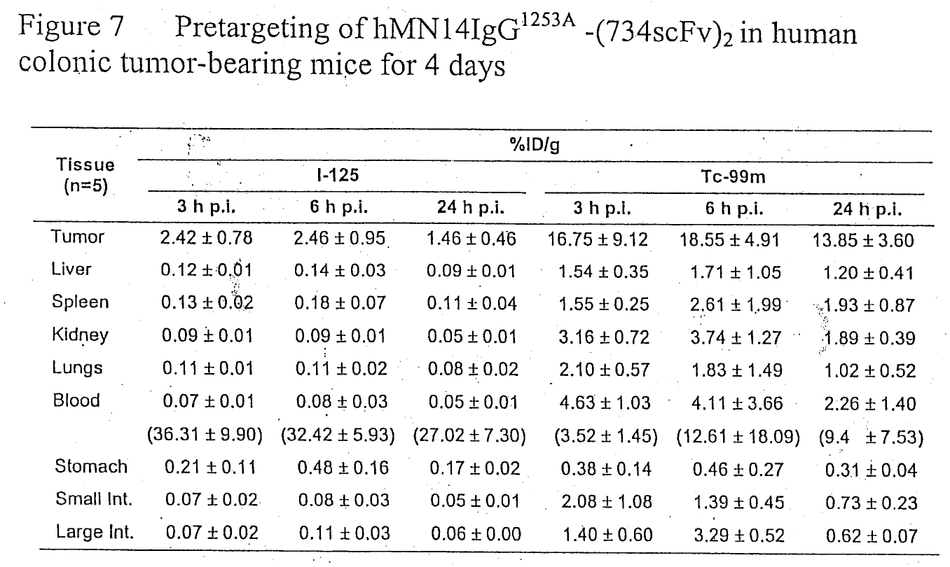

FIGS. 5 and 6 show data for pretargeting methods involving the parent bsAb, ¹²⁵I-hMN-14IgG-(734scFv)₂. FIG. 7 shows data for pretargeting methods involving the mutant bsAb, 125-hMN-14IgG^{I253}A-(734scFv)₂. The ¹²⁵I-label allows for a determination of the amount of bsAb present in different regions of the body. A comparison of the data in FIGS. 5 and 7 shows that the mutant bsAb cleared the body faster than the parent bsAb. For example, after pretargeting with parent bsAb for 4 days (FIG. 5), and 3 hours post injection of IMP-192, the %ID/g for tumor and blood was 19.21±7.318 and 3.73±0.75, respectively. In contrast, after pretargeting with mutant bsAb for 4 days (FIG. 5), and 3 hours post injection of IMP-192, the %ID/g for tumor and blood was 2.42±0.78 and 0.07±0.01, respectively.

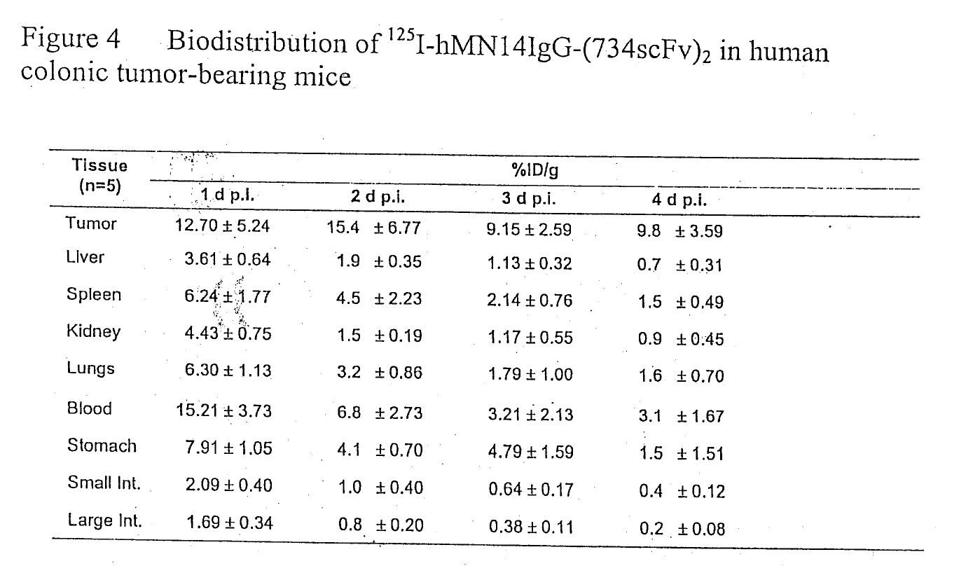

A comparison of the tumor-to-blood ratios of ¹²⁵I in FIGS. 5 and 7 (see entry under “Blood” in FIGS. 5 and 7) demonstrates that a higher signal-to-background can be achieved with the mutant bsAb. Even after 6 days of pretargeting with parent bsAb (see FIG. 4), the tumor-to-blood ratio is much less than after 4 days of pretargeting with mutant bsAb.

The ^{99m}Tc-label allows for a determination of the amount of targetable construct present in different regions of the body. A comparison of the %ID/g of IMP-192 (^{99m}Tc-labeled targetable construct) shows that the tumor-to-blood ratio is much greater for the pretargeting methods with mutant bsAb. This result illustrates that less targetable construct is trapped in the blood in pretargeting methods involving a mutant bsAb. When the parent bsAb is used (see FIGS. 5 and 6) the ^{99m}Tc-labeled targetable construct is trapped in the blood, rather than appearing at the tumor site. Therefore, low tumor-to-blood ratios are observed. For example, the tumor-to-blood ratio of ⁹⁹Tc-labeled targetable construct is shown in FIG. 5 (parent bsAb) in the left hand side, under “Blood”. Three hours post injection, the tumor-to-blood ratio is 0.24±0.05. In contrast, FIG. 5 (mutant bsAb) shows the tumor to blood ratio three hours post injection is 3.52±1.45.

IX. Other Applications

The present invention encompasses the use of the mutant bsAb and a therapeutic agent associated with the linker moieties discussed above in intraoperative, intravascular, and endoscopic tumor and lesion detection, biopsy and therapy as described in U.S. Pat. Nos. 5,716,595 and 6,096,289.

The mutant bsAb of the present invention can be employed not only for therapeutic or imaging purposes, but also as aids in performing research in vitro. For example, the bsAbs of the present invention can be used in vitro to ascertain if a targetable construct can form a stable complex with one or more bsAbs. Such an assay would aid the skilled artisan in identifying targetable constructs which form stable complexes with bsAbs. This would, in turn, allow the skilled artisan to identify targetable constructs which are likely to be superior as therapeutic and/or imaging agents.

The assay is advantageously performed by combining the targetable construct in question with at least two molar equivalents of a mutant bsAb. Following incubation, the mixture is analyzed by size-exclusion HPLC to determine whether or not the construct has bound to the bsAb. Alternatively, the assay is performed using standard combinatorial methods wherein solutions of various bsAbs are deposited in a standard 96 well plate. To each well, is added solutions of targetable construct(s). Following incubation and analysis, one can readily determine which construct(s) bind(s) best to which bsAb(s).

It should be understood that the order of addition of the mutant bsAb to the targetable construct is not crucial; that is, the mutant bsAb may be added to the construct and vice versa. Likewise, neither the mutant bsAb nor the construct needs to be in solution; that is, they may be added either in solution or neat, whichever is most convenient. Lastly, the method of analysis for binding is not crucial as long as binding is established. Thus, one may analyze for binding using standard analytical methods including, but not limited to, FABMS, high-field NMR or other appropriate method in conjunction with, or in place of, size-exclusion HPLC.

### X. EXAMPLES

Materials And Methods

Designing and Construction of 734scFv

734scFv was designed to have the configuration of sL-Vλ-L-V_{H}, where sL is a short flexible linker, Gly-Gly-Gly-Ser (Coloma & Morrison, Nat. Biotechnol. 15:159-163 (1997)), serving as the linkage between hMN-14 IgG heavy chain and 734scFv, and L is a long linker between the Vλ and V_{H} of 734 composed of three repeats of Gly-Gly-Gly-Gly-Ser, (Huston, Levinson, et al. PNAS 85:5879-5883 (1988)). Primer pairs 734V_{L}scFv5′(Cys)/734VLscFv3′ and 734V_{H}scFv5′/734V_{H}scFv3′(SacI) were used to amplify respective V₁ and V_{H} sequences of 734. The resulting DNA products were assembled into 734scFv gene by restriction enzyme digestion and ligation and the sequence was confirmed by DNA sequencing.

734VLSCFV5′(Cys)
5′-TT CTC TCT GCA GAG CCC AAA TCT TGT GGT GGC GGT
TCA CAG CTG GTT GTG ACT CAG-3′
734VLSCFV3′
5′-A GCC TCC GCC TCC TGA TCC GCC ACC TCC TAA GAT
CTT CAG TTT GGT TCC-3′
734VHSCFV5′
5′-CC GGA GGC GGT GGG AGT GAG GTG AAA CTG CAG
GAG-3′
734VHSCFV3′(SacI)
5′-AA CCT TGA GCT CGG CCG TCG CAC TCA TGA GGA GAC
GGT GAC CG-3′

Construction of the Expression Vector for hMN-14IgG-(734scFv)₂

To link 734scFv to the C-terminal end of human heavy constant chain (HC), a new pair of primers, 734scFv2-5′ and 734scFv-3′, was synthesized and used to amplify the DNA encoding 734scFv. The primer 734scFv2-5′ provided the correct sequence for inframe linking 734scFv to the C-terminal end of human HC. The resulting DNA fragment was ligated to human HC sequence, forming a construct encoding HC-734scFv. The DNA fragment encoding normal human HC in the expression vector for hMN-14, hMN-14pdHL2, was then replaced by the HC-734scFv fragment, resulting in the expression vector for the fusion construct, hMN-14IgG-(734scFv)₂pdHL2.

734scFv2-5′ 5′-TCC CCG GGT AAA GGT GGC GGT TCA CAG CTG-3′

734scFv-3′ 5′-GAG CTC GGC CGT CGC AC-3′

Construction of the Mutant Fusion bsAb, hMN-141IgG^{(I253A)}-(734SCFV)₂

Isoleucine 253 is located in the C_{H}2 domain of human HC chain. To introduce the 1253A mutation into hMN-14IgG-(734scFv)₂, plasmid vector C_{H}1kbpKS, containing an insert DNA fragment encoding C_{H}1 and partial C_{H}2 domains was used in oligonucleotide directed site-specific mutagenesis. An oligonucleotide I253AC_{H}2, which converts the wild type sequence KDTLM²⁵³ISRTPE in the C_{H}2 to KDTLM²⁵³ASRTPE, was designed and synthesized as the mutagenic primer. The mutagenisis was accomplished by using the Sculptor IVM system (Amersham, Arlington Heights, Ill.) according to the manufacturer's specifications. After the sequence had been verified by dideoxy DNA sequencing, the mutated HC fragment was subcloned into hMN-14IgG-(734scFv)₂pdHL2 to replace the corresponding wild type fragment, resulting in the expression vector for the mutant fusion bsAb, hMN-14IgG^{(I253A)}-(734scFv)₂pdHL2.

I1253AC_{H}2 5′-AAG GAC ACC CTC ATG GCT AGC CGG ACC CCT GAG-3′

Expression and Production of bsAbs

The expression vectors were transfected into Sp2/0 cells by electroporation 2-5×10⁶ cells were transfected using ^{˜}30 μg of SalI linearized DNA and plated into 96-well cell culture plates. After 2 days, methotrexate (MTX) at a final concentration of 0.025-0.075 μM was added into the cell culture medium for the selection of transfectants. MTX-resistant colonies emerged in 2-3 weeks and were screened by ELISA for secretion of human IgG. Briefly, cell culture supernatants from the surviving colonies were incubated in microwells of ELISA plate coated with goat anti-human IgG F(ab′)₂ specific antibody for 1 h. A peroxidase-conjugated goat anti-human IgG Fc fragment specific antibody was then added and incubated in the wells for 1 h. The presence of human IgG in the supernatant was revealed by addition of the substrate solution containing 0.4 mg/ml of o-phenylenediamine dihydrochloride and 0.0125% H₂O₂. From the positive clones, the best Ab-producers were determined, selected and further expanded. hMN-14IgG-(734scFv)₂ and hMN-14IgG^{(I253A)}-(734scFv)₂ were purified from cell culture supernatant by affinity chromatography on either Protein A or DTPA column.

Synthesis of Ac-Lys(DTPA)-Tyr-Lys(DTPA)-Lys(TscG-Cys-)-NH2 (IMP 192):

The first amino acid, Aloc-Lys(Fmoc)-OH was attached to 0.2 l mmol Rink amide resin on the peptide synthesizer followed by the addition of the Tc-99m ligand binding residues Fmoc-Cys(Trt)-OH and TscG to the side chain of the lysine using standard Fmoc automated synthesis protocols to form the following peptide: Aloc-Lys(TscG-Cys(Trt)-rink resin. The Aloc group was then removed by treatment of the resin with 8 mL of a solution containing 100 mg Pd[P(Ph)₃]₄ dissolved in 10 mL CH₂Cl₂, 0.75 mL glacial acetic acid and 2.5 ml diisopropylethyl amine. The resin mixture was then treated with 0.8 ml tributyltin hydride and vortex mixed for 60 min. The peptide synthesis was then continued on the synthesizer to make the following peptide: Lys(Aloc)-Tyr-Lys(Aloc)-Lys(TscG-Cys-)-rink resin. The N-terminus was acetylated by vortex mixing the resin for 60 mm with 8 mL of a solution containing 10 mL DMF, 3 mL acetic anhydride, and 6 mL diisopropylethylamine. The side chain Aloc protecting groups were then removed as described above and the resin treated with piperidine using the standard Fmoc deprotection protocol to remove any acetic acid which may have remained on the resin.

Activated DTPA and DTPA Addition: The DTPA, 5 g, was dissolved in 40 mL 1.0 M tetrabutylammonium hydroxide in methanol. The methanol was removed under hi-vacuum to obtain a viscous oil. The oil was dissolved in 50 mL DMF and the volatile solvents were removed under hi-vacuum on the rotary evaporator. The DMF treatment was repeated two more times. The viscous oil was then dissolved in 50 ml DMF and mixed with 5 g HBTU. An 8 ml aliquot of the activated DTPA solution was then added to the resin which was vortex mixed for 14 hr. The DTPA treatment was repeated until the resin gave a negative test for amines using the Kaiser test.

Cleavage and Purification: The peptide was then cleaved from the resin by treatment with 8 ml of a solution made from 30 ml TFA, 1 ml triisopropylsilane, and 1 ml ethanedithiol for 60 mm. The crude cleaved peptide was precipitated by pouring into 30 ml ether and was collected by centrifugation. The peptide was then purified by reverse phase HPLC using a 4×30 cm Waters preparative C-18 Delta-Pak column (15 μm, 100 Å). The HPLC fractions were collected and lyophilized to obtain a fraction which contained the desired product by ESMS (MH±1590).

Kit Formulation: The peptide was formulated into lyophilized kits which contained 78 μg of the peptide, 0.92 mg non-radioactive InCl₃, 100 μg stannous chloride, 3 mg gentisic acid, and HPCD (10% on reconstitution).

Radiolabeling

60 μg of antibody protein was labeled with 1-125 using the chloramine-T method (Greenwood, Hunter, et al., Biochem. J. 89 11-123 (1963)) and purified using NAP-5 disalting column (Pharmacia, Piscataway, N.J.).

To prepare Tc-99m labeled IMP-192, a kit containing 50 μg IMP-192 was reconstituted with 1.5 ml of a saline solution containing 20 mCi pertechnetate. The reconstituted kit was incubated at room temperature for 10 min and then heated for 15 min in a boiling water bath.

### Example 1

### Biodistribution ¹²⁵I-hMN-14IgG^{I253A}-(734scFv)₂ and ¹²⁵I-hMN-14IgG-(734scFV)₂ in Human Colonic Tumor-Bearing Mice

Experimental Procedure

Simple biodistribution patterns of the ¹²⁵I-hMN-14IgG-(734scFv)₂ and ¹²⁵I-hMN-14IgG^{I253A}-(734scFv)₂ were evaluated. Groups of nude female mice bearing GW39 human colonic cancer xenografts received i.v. injections of 20 μg (5 μCi)/mouse of a ¹²⁵I-labeled parent or mutant bsAb. Mice were euthanized at designed postinjection time points and their organs were removed, weighted and counted for I-125 radioactivity.

The GW-39 human colonic tumor cell line was propagated as serial, subcutaneous xenografts in nude mice as described elsewhere (Tu, et al. Tumour Biology 9:212-220 (1988)).

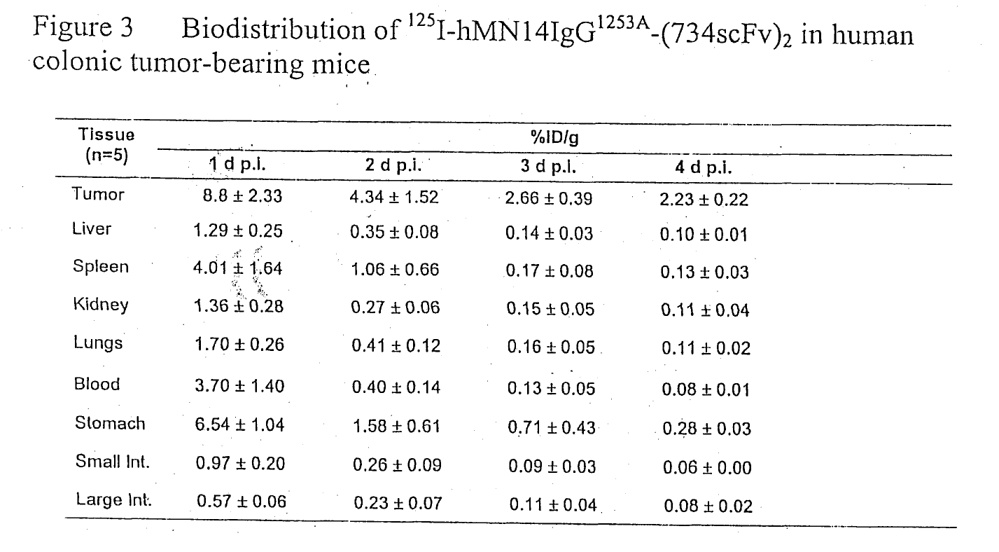

Results The tumor and normal tissue biodistribution of ¹²⁵I-labeled hMN-14IgG-(734scFv)₂ and hMN-14IgG^{I253A}(734scFv)₂ mutant was examined in human colonic tumor-bearing mice 1, 2, 3 and 4 days postinjection. The results are presented in FIGS. 3 and 4 wherein data are expressed as a median percentage of injected dose per gram (% ID/g).

The tumor uptake of hMN-14IgG^{I253A}(734scFv)₂ was significantly lower than that of hMN-14IgG-(734scFv)₂. This accelerated rate of clearance of hMN-14IgG^{I253A}(734scFv)₂ is also seen in normal tissues such as liver, spleen, kidney, lungs, stomach, small intestine, large intestine and blood. See FIGS. 3 and 4. The accelerated clearance of hMN-14IgG^{I253A}(734scFv)₂ produced higher tumor-to-organ ratios for many normal tissues, such as liver, spleen, kidney, lungs, stomach, small intestine, large intestine and blood. Additionally, the tumor-to-blood ratio for the hMN-14IgG^{I253A}(734scFv)₂ mutant increased at a much faster from one to four days postinjection as compared to the tumor/blood ratio for hMN-14IgG-(734scFv)₂.

### Example 2

### Pretargeting of ¹²⁵I-hMN-14IgG^{I253A}-(734scFV)₂ and ¹²⁵I-hMN-14IgG-(734scFV)₂ in human colonic tumor-bearing mice

Experimental Procedure

Pretargeting biodistribution patterns of mutant and parent bsAbs were evaluated. Groups of nude female mice bearing GW39 human colonic cancer xenografts received i.v. injections of 20 μg (5 μCi)/mouse of a ¹²⁵I-labeled mutant or parent bsAb. Following the injection of mutant or parent bsAb, a predetermined clearance time was allowed for bsAb to localize to tumor sites and be removed from circulation. The ^{99m}Tc-labeled divalent DTPA peptide, IMP-192, was then administered i.v. The mice were sacrificed at various time points of postinjection of the peptide and their organs were removed, weighted and counted for both I-125 and Tc-99m radioactivities.

The GW-39 human colonic tumor cell line was propagated as serial, subcutaneous xenografts in nude mice as described elsewhere (Tu, et al. Tumour Biology 9:212-220 (1988)).

Results

The tumor and normal tissue biodistribution of ¹²⁵¹I-labeled hMN-14IgG^{I253A}-(734scFv)₂ and ¹²⁵I-labeled hMN-14IgG-(734scFv)₂ was examined in human colonic tumor-bearing mice 3, 6 and 24 hours postinjection of ^{99m}Tc-labeled divalent DTPA peptide, IMP-192. Prior to injection of IMP-192 pretargeting with mutant or parent bsAb was performed for four days. The tumor and normal tissue biodistribution of ¹²⁵I-labeled mutant and parent bsAb are shown in FIGS. 5-7, wherein data are expressed as a median percentage of injected dose per gram (% ID/g). Additionally, the tumor and normal tissue biodistribution of IMP-192 (^{99m}Tc-labeled divalent DTPA peptide) are shown in FIGS. 5-7. Accelerated clearance of the mutant bsAb is observed. Additionally, higher tumor-to-blood ratios are observed after pretargeting with mutant bsAb as compared to pretargeting with parent bsAb. It is noted that more DTPA-peptide was trapped in the blood after pretargeting with the parent fusion protein then after pretargeting with the mutant fusion protein.

It will be apparent to those skilled in the art that various modifications and variations can be made to the compositions and processes of this invention. Thus, it is intended that the present invention cover such modifications and variations, provided they come within the scope of the appended claims and their equivalents.

The disclosure of all publications cited above are expressly incorporated herein by reference in their entireties to the same extent as if each were incorporated by reference individually.

### Example 3

### Binding of In-DTPA Containing Peptides to hMN-14IgG^{I253A}-(734scFV)₂

The binding of In-DTPA peptides to the anti-In-DTPA antibody hMN-14IgG^{(I253A)}-(734scFv)₂ was examined by size exclusion HPLC and by affinity blocking studies using the Biacore X:

Binding Analysis Using HPLC

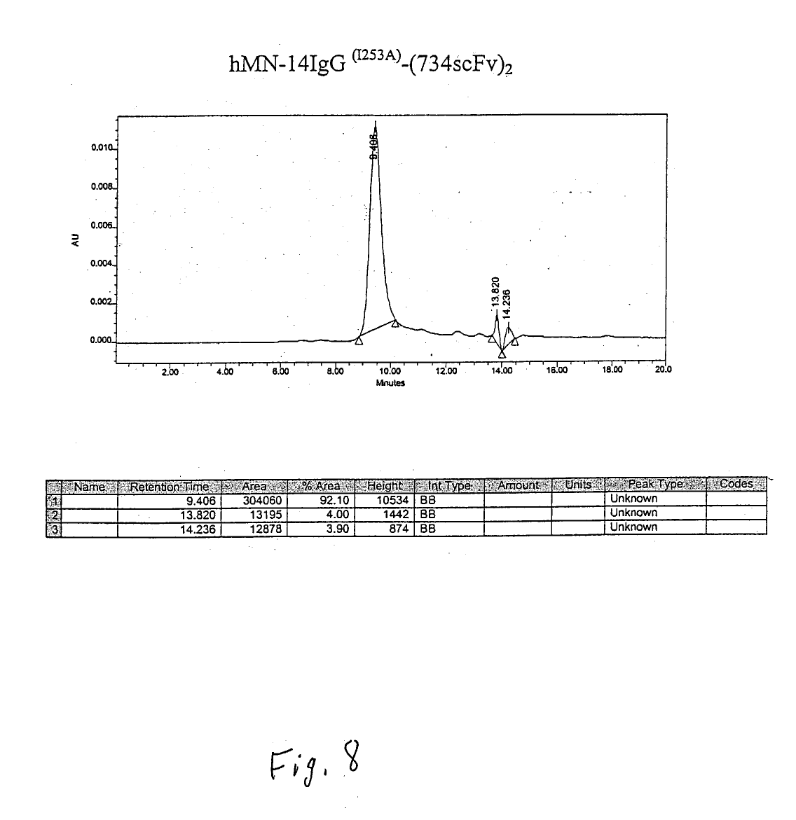

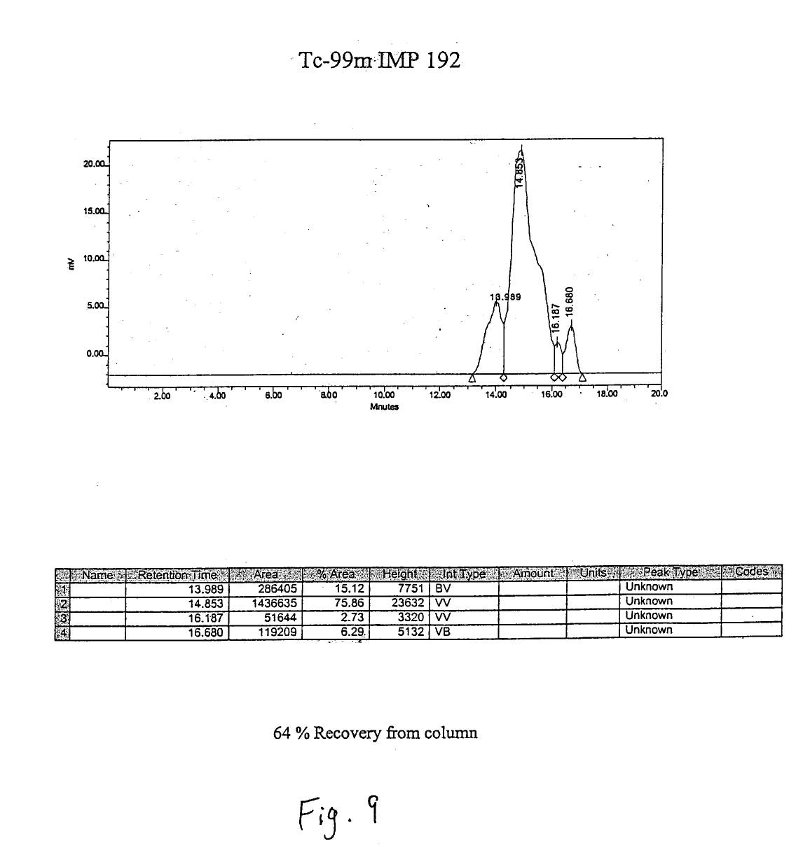

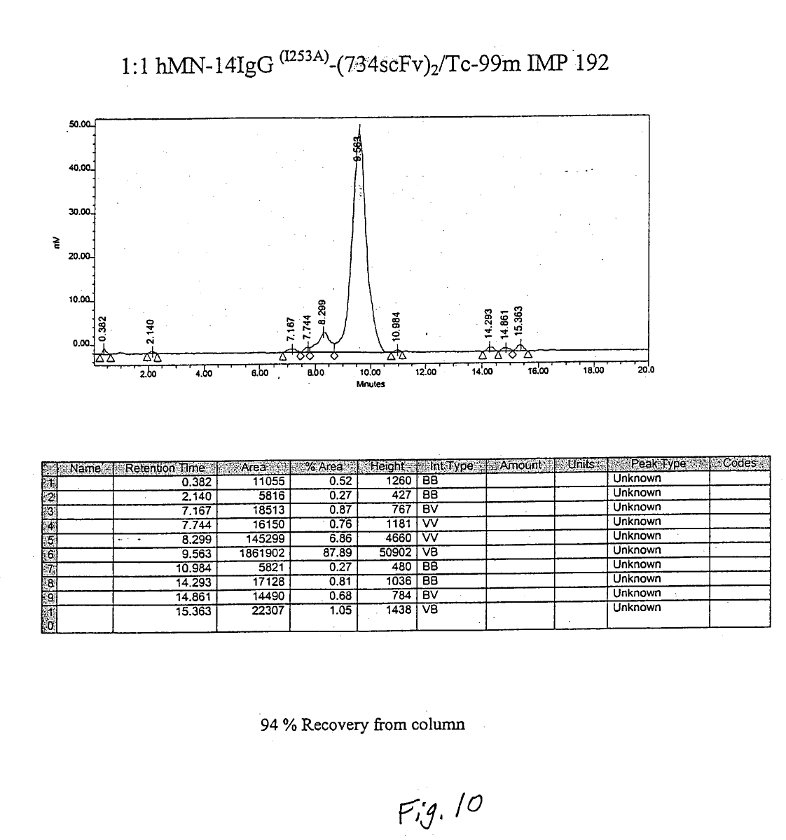

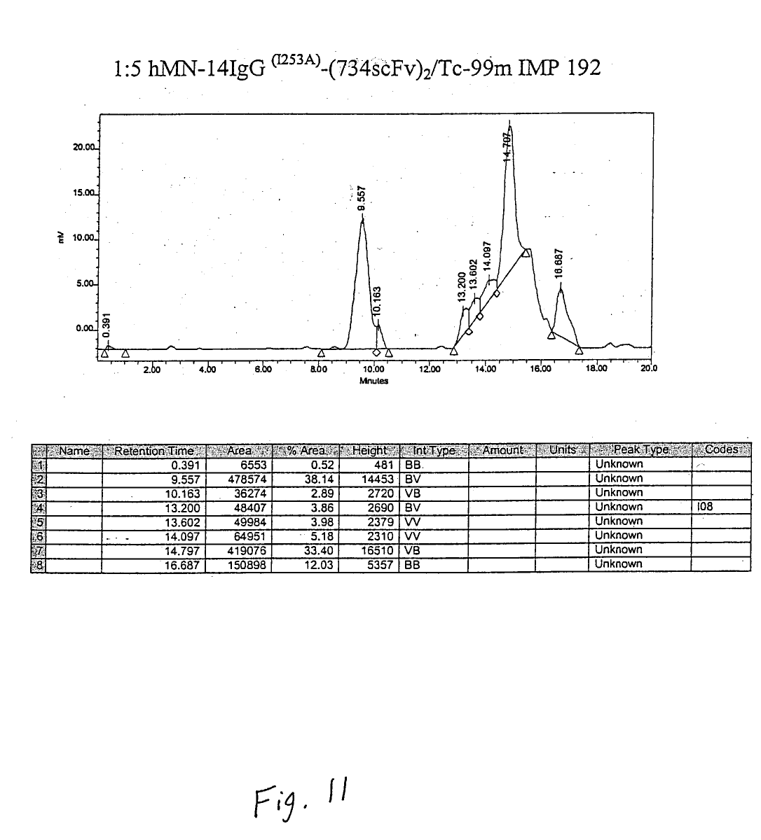

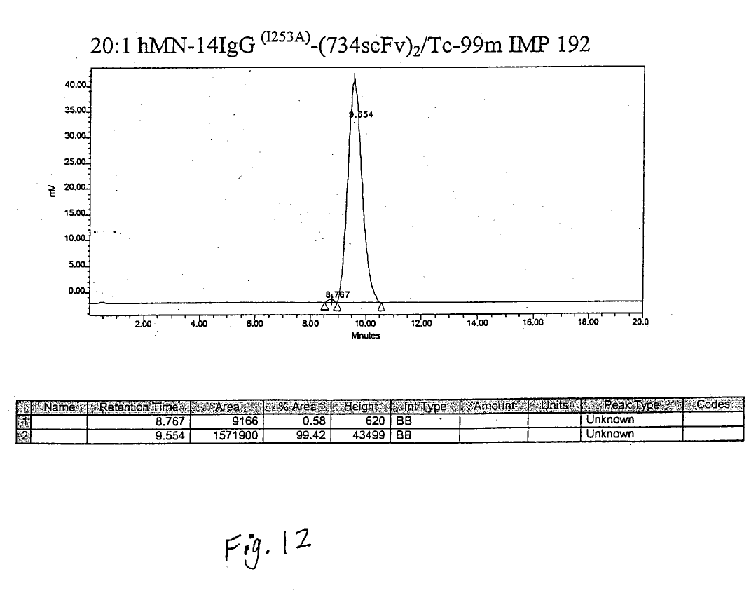

An IMP 192 kit was labeled with Tc-99m 20.9 mCi. Aliquots from the kit were diluted and mixed with hMN-14IgG^{(I253A)}-(734scFv)₂ in the following molar ratios (Peptide/ab) 1:5, 1:1, and 20:1. The peptide/antibody mixtures, the peptide alone and the antibody alone were examined on a Bio-Sil SEC 250 300 mm×7.8 mm HPLC column elluted at 1 mL/min with 0.2 M phosphate buffer pH 6.8. The HPLC traces (FIGS. 8-12 show essentially only one peptide/antibody complex is formed. A known standard of hMN-14IgG^{I253A}-(734scFv)₂ eluted from the column at about 9.41 minutes (FIG. 8). A known standard of Tc-99m IMP 192 eluted from the column at about 14.85 minutes (FIG. 9). When a 1:1 mixture of hMN-14IgG^{I253A}-(734scFv)₂ to Tc-99m IMP 192 were applied to the column, only one peak was observed at about 9.56 minutes (FIG. 10). In contrast, when a 1:5 mixture of hMN-14IgG^{I253A}-(734scFv)₂ to Tc-99m IMP 192 was applied to the column, two major peaks were observed, one at about 9.56 minutes (hMN-14IgG^{I253A}-(734scFv)₂) and the other at about 14.80 minutes (Tc-99m IMP 192) (FIG. 11). When a 20:1 mixture of hMN-14IgG^{I253A}-(734scFv)₂ to Tc-99m IMP 192 was applied to the column, only one peak was observed at 9.56 minutes (FIG. 12).

### Example 4

### Clinical Examples

Example 4A. A patient with a colon polyp has the polyp removed, and it is found to be malignant. CAT scan fails to demonstrate any tumor, but the patient after three months has a rising blood CEA level. The patient is given 10 mg of hMN14-IgG[734-scFv]2 by i.v. infusion. Three days later the patient is given the bivalent peptide IMP 192 labeled with 40 mCi of Tc-99m. The next day the patient undergoes radioscintigraphy, and a single locus of activity is observed in a node close to the site of the resected polph. The node is resected, and patient remains free of disease for the next 10 years.

### Example 4B

A patient with colon carcinoma undergoes resection of the primary tumor. Two years later the patient presents with a rising CEA blood level, and CAT scan demonstrates multiple small metastasis in the liver, which cannot be resected. The patient is given 100 mg of hMN14-IgG[734-scFv]2 by i.v. infusion. After 3 days the patient if given the bivalent-DTPA peptide, IMP 156, labeled with 160 mCi of 1-131 by i.v. infusion. The CEA blood level slowly drops into the normal range. CAT scan demonstrates resolution of several of the metastasis, and the remaining lesions fail to grow for the next 9 months.

It will be apparent to those skilled in the art that various modifications and variations can be made to the compositions and processes of this invention. Thus, it is intended that the present invention cover such modifications and variations, provided they come within the scope of the appended claims and their equivalents.

The disclosure of all publications, patents, and patent applications cited above are expressly incorporated herein by reference in their entireties to the same extent as if each were incorporated by reference individually.

In the Specificati n:

Please amend the specification as shown:

Please delete the paragraph on page 4, lines 16-19, and replace it with the following paragraph:

FIG. 1 shows the heavy chain cDNA (SEQ ID NO: 1) and amino acid (SEQ ID NO: 2) sequences of hMN-14. The V_{H}, C_{H}1, Hinge, C_{H}2 and C_{H}3 regions are shown. The isoleucine at amino acid position 274 corresponds to isoleucine 253 according to the numbering system of Edelman, et al. See Edelman et al. Biochemistry 63, 78-85 (1969).

Please delete the paragraph on page 4, lines 20-21, and replace it with the following paragraph:

FIG. 2 shows the light chain cDNA (SEQ ID NO: 3) and amino acid (SEQ ID NO: 4) sequences of hMN-14. The V_{K} and C_{K} regions are shown.

Please delete the paragraph text on page 17, line 19 to page 18, line 2, and replace it with the following paragraph text:

Peptides having as few as two amino-acid residues may be used, preferably two to ten residues, if also coupled to other moieties, such as chelating agents. The linker should be a low molecular weight conjugate, preferably having a molecular weight of less than 50,000 daltons, and advantageously less than about 20,000 daltons, 10,000 daltons or 5,000 daltons, including the metal ions in the chelates. For instance, the known peptide DTPA-Tyr-Lys(DTPA)-OH (wherein DTPA is diethylenetriaminepentaacetic acid) has been used to generate antibodies against the indium-DTPA portion of the molecule. However, by use of the non-indium-containing molecule, and appropriate screening steps, new Abs against the tyrosyl-lysine dipeptide can be made. More usually, the antigenic peptide will have four or more residues, such as the peptide DOTA-Phe-Lys(HSG)-Tyr-Lys(HSG)-NH₂ (SEQ ID NO: 5), wherein DOTA is 1,4,7,10-tetraazacyclododecanetetraacetic acid and HSG is the histamine succinyl glycyl group of the formula:

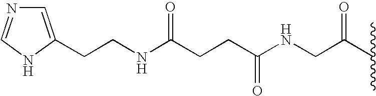

Please delete the paragraph on page 18, lines 3-4, and replace it with the following paragraph:

The non-metal-containing peptide may be used as an immunogen, with resultant Abs screened for reactivity against the Phe-Lys-Tyr-Lys (SEQ ID NO: 5) backbone.

Please delete the paragraph on page 19, lines 1-5, and replace it with the following paragraph:

The targetable construct may be monovalent or bivalent, with bivalent peptides being the preferred peptide. One exemplary targetable construct is IMP 192 (Ac-Lys(DTPA)-Tyr-Lys(DTPA)-Lys(TscG-Cys-)-NH₂) (SEQ ID NO: 6). IMP 192 binds both Tc-99m and In-111 for diagnosis, and Re-188 and Re-186 for therapy. IMP 192 also binds bivalent DTPA-peptides with tyrosine.

Please delete the paragraph on page 20, line 29 to page 21, line 15, and replace it with the following paragraph:

Chelators such as those disclosed in U.S. Pat. No. 5,753,206, especially thiosemi-carbazonylglyoxylcysteine(Tscg-Cys) and thiosemicarbazinyl-acetylcysteine (Tsca-21 Cys) chelators are advantageously used to bind soft acid cations of Tc, Re, Bi and other transition metals, lanthamides and actinides that are tightly bound to soft base ligands, especially sulfur- or phosphorus-containing ligands. It can be useful to link more than one type of chelator to a peptide, e.g., a DTPA or similar chelator for, say In(III) cations, and a thiol-containing chelator, e.g., Tscg-Cys, for Tc cations. Because antibodies to a di-DTPA hapten are known (Barbet '395, supra) and are readily coupled to a targeting antibody to form a bsAb, it is possible to use a peptide hapten with cold diDTPA chelator and another chelator for binding a radioisotope, in a pretargeting protocol, for targeting the radioisotope. One example of such a peptide is Ac-Lys(DTPA)-Tyr-Lys(DTPA)-Lys(Tscg-Cys-)-NH₂ (SEQ ID NO: 6). This peptide can be preloaded with In(III) and then labeled with 99m-Tc cations, the In(III) ions being preferentially chelated by the DTPA and the Tc cations binding referentially to the thiol-containing Tscg-Cys. Other hard acid chelators such as NOTA, DOTA, TETA and the like can be substituted for the DTPA groups, and Mabs specific to them can be produced using analogous techniques to those used to generate the anti-di-DTPA Mab.

Please delete the paragraph text on page 21, lines 23-28 and replace it with the following paragraph text:

Preferred chelators include NOTA, DOTA and Tscg and combinations thereof. These chelators have been incorporated into a chelator-peptide conjugate motif as exemplified in the following constructs:

| | |
|---|---|
| (a) | DOTA-Phe-Lys(HSG)-D-Tyr-LYS(HSG)-NH₂; |
| (b) | DOTA-Phe-Lys(HSG)-Tyr-Lys(HSG)NH₂ (SEQ ID NO: 5); |
| (c) | Ac-Lys(HSG)D-Tyr-Lys(HSG)-Lys(Tscg-Cys)-NH₂; |
| (d) | 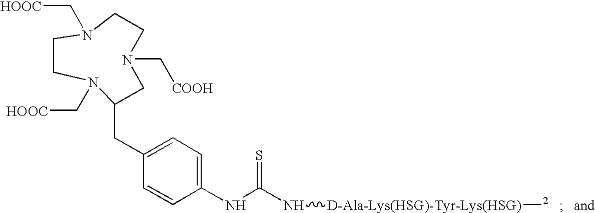 |
| (e) | 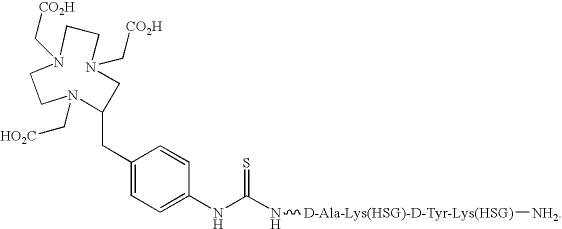 |

Please delete the paragraph on page 22, line 3 to page 23, line 2 and replace it with the following paragraph:

Chelators are coupled to the linker moieties using standard chemistries which are discussed more fully in the working Examples below. Briefly, the synthesis of the peptide Ac-Lys(HSG)D-Tyr-Lys(HSG)-Lys(Tscg-Cys-)-NH₂ was accomplished by first attaching Aloc-Lys(Fmoc)-OH to a Rink amide resin on the peptide synthesizer. The protecting group abbreviations “Aloc” and “Fmoc” used herein refer to the groups allyloxycarbonyl and fluorenylmethyloxy carbonyl. The Fmoc-Cys(Trt)-OH and TscG were then added to the side chain of the lysine using standard Fmoc automated synthesis protocols to form the following peptide: Aloc-Lys(Tscg-Cys(Trt)-rink resin. The Aloc group was then removed. The peptide synthesis was then continued on the synthesizer to make the following peptide: (Lys(Aloc)-D-Tyr-Lys(Aloc)-Lys(Tscg-Cys(Trt)-)-rink resin. Following N-terminus acylation, and removal of the side chain Aloc protecting groups. The resulting peptide was then treated with activated N-trityl-HSG-OH until the resin gave a negative test for amines using the Kaiser test. See Karacay et al. Bioconjugate Chem. 11:842-854 (2000). The synthesis of Ac-Lys(HSG)D-Tyr-Lys(HSG)-Lys(Tscg-Cys-)-NH₂, as well as the syntheses of DOTA-Phe-Lys(HSG)-D-Tyr-Lys(HSG)-NH₂; and DOTA-Phe-Lys(HSG)-Tyr-Lys(HSG)-NH₂ (SEQ ID NO: 5) are described in greater detail below.

Please delete the paragraph on page 27, line 24 to page 28, line 10 and replace it with the following paragraph:

A similar strategy can be employed to obtain high-affinity scFv. See, e.g., Vaughn et al., Nat. Biotechnol., 14: 309-314 (1996). An scFv library with a large repertoire can be constructed by isolating V-genes from non-immunized human donors using PCR primers corresponding to all known V_{H}, V and V gene families. Following amplification, the V and V pools are combined to form one pool. These fragments are ligated into a phagemid vector. The scFv linker, (Gly₄, Ser)₃,(SEQ ID NO: 7) is then ligated into the phagemid upstream of the V_{L} fragment. The V_{H} and linker-V_{L} fragments are amplified and assembled on the J_{H} region. The resulting V_{H}-linker-V_{L} fragments are ligated into a phagemid vector. The phagemid library can be panned using filters, as described above, or using immunotubes (Nunc; Maxisorp). Similar results can be achieved by constructing a combinatorial immunoglobulin library from lymphocytes or spleen cells of immunized rabbits and by expressing the scFv constructs in P. pastoris. See, e.g., Ridder et al., Biotechnology, 13: 255-260 (1995). Additionally, following isolation of an appropriate scFv, antibody fragments with higher binding affinities and slower dissociation rates can be obtained through affinity maturation processes such as CDR3 mutagenesis and chain shuffling. See, e.g., Jackson et al., Br. J. Cancer, 78: 181-188 (1998); Osbourn et al., Immunotechnology, 2: 181-196 (1996).

Please delete the paragraph on page 40, lines 3-20 and replace it with the following paragraph:

Designing and Construction of 734scFv

734scFv was designed to have the configuration of sL-Vλ-L-V_{H}, where sL is a short flexible linker, Gly-Gly-Gly-Ser (SEQ ID NO: 8) (Coloma & Morrison, Nat. Biotechnol. 15:159-163 (1997)), serving as the linkage between hMN-14 IgG heavy chain and 734scFv, and L is a long linker between the Vλ and V_{H} of 734 composed of three repeats of Gly-Gly-Gly-Gly-Ser, (SEQ ID NO: 9) (Huston, Levinson, et al. PNAS 85:5879-5883 (1988)). Primer pairs 734V_{L}scFv5′(Cys)/734VLscFv3′ and 734V_{H}scFv5′/734V_{H}scFv3′(SacI) were used to amplify respective V_{I} and V_{H} sequences of 734. The resulting DNA products were assembled into 734scFv gene by restriction enzyme digestion and ligation and the sequence was confirmed by DNA sequencing.

| | | |
|---|---|---|
| 734VLSCFV5′(Cys) | 5′-TT CTC TCT GCA GAG CCC AAA TCT TGT GGT GGC | (SEQ ID NO:10) |
| | GGT | |
| | TCA CAG CTG GTT GTG ACT CAG-3′ | |
| 734VLSCFV3′ | 5′-A GCC TCC GCC TCC TGA TCC GCC ACC TCC TAA GAT | (SEQ ID NO:11) |
| | CTT CAG TTT GGT TCC-3′ | |
| 734VHSCFV5′ | 5′-CC GGA GGC GGT GGG AGT GAG GTG AAA CTG CAG | (SEQ ID NO:12) |
| | GAG-3′ | |
| 734VHSCFV3′(SacI) | 5′-AA CCT TGA GCT CGG CCG TCG CAC TCA TGA GGA | (SEQ ID NO:13) |
| | GAC | |
| | GGT GAC CG-3′ | |

Please delete the paragraph on page 40, line 21 to page 41, line 6 and replace it with the following paragraph:

Construction of the Expression Vector for hMN-14IgG-(734scFv)₂

To link 734scFv to the C-terminal end of human heavy constant chain (HC), a new pair of primers, 734scFv2-5′ and 734scFv-3′, was synthesized and used to amplify the DNA encoding 734scFv. The primer 734scFv2-5′ provided the correct sequence for inframe linking 734scFv to the C-terminal end of human HC. The resulting DNA fragment was ligated to human HC sequence, forming a construct encoding HC-734scFv. The DNA fragment encoding normal human HC in the expression vector for hMN-14, hMN-14pdHL2, was then replaced by the HC-734scFv fragment, resulting in the expression vector for the fusion construct, hMN-14IgG-(734scFv)₂pdHL2.

| | | |
|---|---|---|
| 734SCFV2-5′ | 5′-TCC CCG GGT AAA GGT GGC GGT TCA CAG CTG-3′ | (SEQ ID NO:14) |
| 734SCFV-3′ | 5′-GAG CTC GGC CGT CGC AC-3′ | (SEQ ID NO:15) |

Please delete the paragraph on page 41, lines 7-18 and replace it with the following paragraph:

Construction of the Mutant Fusion bsAb, hMN-14IgG_{(I253A)}-(734scFv)₂

Isoleucine 253 is located in the C_{H}2 domain of human HC chain. To introduce the I253A mutation into hMN-14IgG-(734scFv)₂, plasmid vector C_{H}1 kbpKS, containing an insert DNA fragment encoding C_{H}1 and partial C_{H}2 domains was used in oligonucleotide directed site-specific mutagenesis. An oligonucleotide I253AC_{H}2, which converts the wild type sequence KDTLM²⁵³ISRTPE (SEQ ID NO: 16) in the C_{H}2 to KDTLM²⁵³ASRTPE (SEQ ID NO: 17), was designed and synthesized as the mutagenic primer. The mutagenisis was accomplished by using the Sculptor IVM system (Amersham, Arlington Heights, Ill.) according to the manufacturer's specifications. After the sequence had been verified by dideoxy DNA sequencing, the mutated HC fragment was subcloned into hMN-14IgG-(734scFv)₂pdHL2 to replace the corresponding wild type fragment, resulting in the expression vector for the mutant fusion bsAb, hMN-14IgG_{(I253A)}-(734scFv)₂pdHL2.

| | | |
|---|---|---|
| I253AC_{H}2 | 5′-AAG GAC ACC CTC ATG GCT AGC CGG ACC CCT GAG-3′ | (SEQ ID NO:18) |

Please delete the paragraph on page 42, lines 9-23 and replace it with the following paragraph:

Synthesis of Ac-Lys(DTPA)-Tyr-Lys(DTPA)-Lys(TscG-Cys-)-NH₂ (SEQ ID NO: 6) (IMP 192):

The first amino acid, Aloc-Lys(Fmoc)-OH was attached to 0.2 l mmol Rink amide resin on the peptide synthesizer followed by the addition of the Tc-99m ligand binding residues Fmoc-Cys(Trt)-OH and TscG to the side chain of the lysine using standard Fmoc automated synthesis protocols to form the following peptide: Aloc-Lys(TscG-Cys(Trt)-rink resin. The Aloc group was then removed by treatment of the resin with 8 mL of a solution containing 100 mg Pd[P(Ph)₃]₄ dissolved in 10 mL CH₂Cl₂, 0.75 mL glacial acetic acid and 2.5 ml diisopropylethyl amine. The resin mixture was then treated with 0.8 ml tributyltin hydride and vortex mixed for 60 min. The peptide synthesis was then continued on the synthesizer to make the following peptide: Lys(Aloc)-Tyr-Lys(Aloc)-Lys(TscG-Cys-)-rink (SEQ ID NO: 6) resin. The N-terminus was acetylated by vortex mixing the resin for 60 mm with 8 mL of a solution containing 10 mL DMF, 3 mL acetic anhydride, and 6 mL diisopropylethylamine. The side chain Aloc protecting groups were then removed as described above and the resin treated with piperidine using the standard Fmoc deprotection protocol to remove any acetic acid which may have remained on the resin.

What is claimed is:

1. A mutant bispecific antibody, comprising at least:
(a) a human hinge constant region from IgG;
(b) two scFvs; and
(c) two Fvs,
wherein said constant region contains one or more amino acid mutations in the C_{H}2 domain.

2. The mutant bispecific antibody of claim 1, wherein said scFvs and said Fvs are CDR-grafted murine scFvs and Fvs.

3. The mutant bispecific antibody of claim 1, wherein said scFvs and said Fvs are humanized or human.

4. The mutant bispecific antibody of claim 1, wherein said hinge constant region contains a mutation in which isoleucine at position 253 is replaced with alanine, or amino acids other than leucine, wherein the amino acid replacement enhances blood clearance equal to, or greater than replacement with alanine.

5. The mutant bispecific antibody of claim 4, wherein said Fvs are derived from hMN14-IgG and said scFvs are 734scFv.

6. The mutant bispecific antibody of claim 1, wherein said scFvs bind a monovalent targetable construct.

7. The mutant bispecific antibody of claim 1, wherein said scFvs bind a divalent targetable construct.

8. The mutant bispecific antibody of claim 1, wherein said Fvs bind to an epitope on a target cell.

9. A method of treating or diagnosing or treating and diagnosing a disease or a condition that may lead to a disease comprising
(A) administering to said subject the mutant bispecific antibody of claim 1, wherein the Fvs are directed to a marker substance associated with the disease or condition;
(B) optionally, administering to said subject a clearing composition, and allowing said composition to clear non-localized antibodies or antibody fragments from circulation; and
(C) administering to said subject a targetable construct comprising a bivalent hapten, wherein both hapten moieties bind to the two scFvs on a single molecule of the mutant bi-specific of claim 1, wherein the targetable construct further comprises a diagnostic or therapeutic cation, and/or one or more chelated or chemically bound therapeutic or diagnostic agents.

10. The method as claimed in claim 9, wherein said mutant bispecific antibody is administered before, during or after the administration of at least one therapeutic agent used to treat the disease or condition.

11. The method as claimed in claim 9, wherein said targetable construct comprises an enzyme and said method further comprises administering to said subject
a) a prodrug, when said enzyme is capable of converting said prodrug to a drug at the target site; or
b) a drug which is capable of being detoxified in said subject to form an intermediate of lower toxicity, when said enzyme is capable of reconverting said detoxified intermediate to a toxic form, and, therefore, of increasing the toxicity of said drug at the target site, or
c) a prodrug which is activated in said subject through natural processes and is subject to detoxification by conversion to an intermediate of lower toxicity, when said enzyme is capable of reconverting said detoxified intermediate to a toxic form, and, therefore, of increasing the toxicity of said drug at the target site.

12. The method of claim 11, wherein said prodrug is selected from the group consisting of epirubicin glucuronide, CPT-11, etoposide glucuronide, daunomicin glucuronide and doxorubicin glucuronide

13. The method of claim 9, wherein said diagnostic agent emits 25 to 4,000 keV gamma particles and/or positrons.

14. The method of claim 9, wherein the diagnostic agent is selected from the group consisting of ¹⁸F, ⁵²Fe, ⁶²Cu, ⁶⁴Cu, ⁶⁷Cu, ⁶⁷Ga, ⁶⁸Ga, ⁸⁶Y, ⁸⁹Zr ^{94m}Tc, ⁹⁴Tc, ^{99m}Tc, ¹¹¹In, ¹²³I, ¹²⁴I, ¹²⁵I, ¹³¹I, ¹⁵⁴⁻¹⁵⁸Gd, ¹⁷⁷Lu, ³²P, 188Re, and ⁹⁰Y or a combination thereof.

15. The method of claim 9, wherein said radioactive isotope is used to perform positron-emission tomography (PET).

16. The method of claim 9, wherein said targetable construct comprises one or more image enhancing agents for use in magnetic resonance imaging (MRI).

17. The method of claim 16, wherein said enhancing agent is selected from the group consisting of Mn, Fe and Gd.

18. The method of claim 9, wherein said targetable construct comprises one or more image enhancing agents for use in ultrasound imaging.

19. The method of claim 18, wherein said targetable construct is a liposome with a bivalent DTPA-peptide covalently attached to the outside surface of the liposome lipid membrane.

20. The method of claim 19, wherein said liposome is gas filled.

21. The method of claim 9, wherein said targetable construct comprises one or more radioactive isotopes useful for killing diseased tissue.

22. The method of claim 21, wherein the energy range of the radioactive isotope is 60 to 700 keV.

23. The method of claim 21, wherein said radioactive isotope is selected from the group consisting of ³²P, ³³P, ⁴⁷Sc, ⁶⁴Cu, ⁶⁷Cu, ⁶⁷Ga, ⁹⁰Y, ¹¹¹Ag, ₁₁₁In, ¹²⁵I, ¹³¹I, ¹⁴²Pr, ¹⁵³Sm, ¹⁶¹Tb, ¹⁶⁶Dy, ¹⁶⁶ Ho, ¹⁷⁷Lu, ¹⁸⁶Re, ¹⁸⁸Re, ¹⁸⁹Re, ²¹²Pb, ²¹²Bi, ²¹³Bi, ²¹¹At, ²²³Ra and ²²⁵Ac or a combination thereof.

24. The method of claim 21, wherein said targetable construct comprises ¹⁰B atoms, and said method further comprises the step of irradiating said boron atoms localized at said diseased tissue, thereby effecting BNCT of said diseased tissue.

25. The method of claim 9, wherein said therapeutic agent is a drug, toxin, hormone, enzyme, immunomodulator, chelator, boron compound, photoactive agent, dye, or radioisotopes.

26. The method of claim 21, wherein said targetable construct comprises one or more toxins.

27. The method of claim 26, wherein said toxin is selected from the group consisting of ricin, abrin, ribonuclease, DNase 1, Staphylococcal enterotoxin-A, pokeweed antiviral protein, gelonin, diphtherin toxin, Pseudomonas exotoxin, and Pseudomonas endotoxin or a combination thereof.

28. The method of claim 9, wherein said targetable construct comprises one or more drugs.

29. The method of claim 28, wherein said drug is selected from the group consisting of nitrogen mustards, ethylenimine derivatives, alkyl sulfonates, nitrosoureas, triazenes, folic acid analogs, anthracyclines, taxanes, COX-2 inhibitors, pyrimidine analogs, purine analogs, antibiotics, enzymes, epipodophyllotoxins, platinum coordination complexes, vinca alkaloids, substituted ureas, methyl hydrazine derivatives, adrenocortical suppressants, antagonists, endostatin, taxols, camptothecins, doxorubicins and their analogs, and a combination thereof.

30. The method of claim 9, wherein the targetable construct comprises one or more agents for photodynamic therapy.

31. The method of claim 30, wherein said agent for photodynamic therapy is a photosensitizer.

32. The method of claim 31, wherein said photosensitizer is selected from the group consisting of benzoporphyrin monoacid ring A (BPD-MA), tin etiopurpurin (SnET2), sulfonated aluminum phthalocyanine (AlSPc) and lutetium texaphyrin (Lutex).

33. The method of claim 9, wherein said targeted tissue is a tumor.

34. The method of claim 11, wherein said targeted tissue is a tumor.

35. The method of claims 33 or 34, wherein said tumor produces or is associated with antigens selected from the group consisting of colon-specific antigen-p (CSAp), carcinoembryonic antigen (CEA), CD4, CD5, CD8, CD14, CD15, CD19, CD20, CD21, CD22, CD23, CD25, CD33, CD37, CD38, CD40, CD40L, CD46, CD52, CD54, CD66a-e, CD74, CD75, CD80, CD126, B7, HLA-DR, Ia, Ii, HM1.24, MUC 1, MUC2, MUC3, MUC4,Tag-72, PSMA, EGP-1, EGP-2, PSA, AFP, HCG, HCG-beta, PLAP, PAP, histone, tenascin, VEGF, P1GF, S10O, EGFR, insulin-like growth factor, HER2/neu, organotropic hormones, oncogene products, and cytokeratin.

36. The method of claims 9 or 11, wherein the mutant bispecific antibody incorporates the Fv of a Class III anti-CEA antibody.

37. The method of claims 9 or 11, wherein the mutant bispecific antibody incorporates the scFv of Mab 679.

38. The method of claims 9 or 11, wherein said disease is an immune dysregulation disease, an autoimmune disease, organ graft rejection, cardiovascular disease, neurological disease or graft vs. host disease.

39. The method of claim 38 wherein said autoimmune disease is selected from the group consisting of acute idiopathic thrombocytopenic purpura, chronic idiopathic thrombocytopenic purpura, dermatomyositis, Sydenham's chorea, myasthenia gravis, systemic lupus erythematosus, lupus nephritis, rheumatic fever, polyglandular syndromes, bullous pemphigoid, diabetes mellitus, Henoch-Schonlein purpura, post-streptococcalnephritis, erythema nodosurn, Takayasu's arteritis, Addison's disease, rheumatoid arthritis, multiple sclerosis, sarcoidosis, ulcerative colitis, erythema multiforme, IgA nephropathy, polyarteritis nodosa, ankylosing spondylitis, Goodpasture's syndrome, thromboangitisubiterans, Sjogren's syndrome, primary biliary cirrhosis, Hashimoto's thyroiditis, thyrotoxicosis, scleroderma, chronic active hepatitis, polymyositis/dermatomyositis, polychondritis, parnphigus vulgaris, Wegener's granulomatosis, membranous nephropathy, amyotrophic lateral sclerosis, tabes dorsalis, giant cell arteritis/polymyalgia, perniciousanemia, rapidly progressive glomerulonephritis and fibrosing alveolitis.

40. The method of claims 9 or 11, wherein said disease caused by a fungus, virus, parasite or bacterium, and the Fv of the mutant bispecific targets the fungus, virus, parasite, or bacterium.

41. The method of claim 40, wherein said virus is selected from the group consisting of human immunodeficiency virus (HIV), herpes virus, cytomegalovirus, rabies virus, influenza virus, hepatitis B virus, Sendai virus, feline leukemia virus, Reo virus, polio virus, human serum parvo-like virus, simian virus 40, respiratory syncytial virus, mouse mammary tumor virus, Varicella-Zoster virus, Dengue virus, rubella virus, measles virus, adenovirus, human T-cell leukemia viruses, Epstein-Barr virus, murine leukemia virus, mumps virus, vesicular stomatitis virus, Sindbis virus, lymphocytic choriomeningitis virus, wart virus and blue tongue virus.

42. The method of claim 40, wherein said bacterium is selected from the group consisting of Anthrax bacillus, Streptococcus agalactiae, Legionella pneumophilia, Streptococcus pyogenes, Escherichia coli, Neisseria gonorrhoeae, Neisseria meningitidis, Pneumococcus, Hemophilis influenzae B, Treponema pallidum, Lyme disease spirochetes, Pseudomonas aeruginosa, Mycobacterium leprae, Brucella abortus, Mycobacterium tuberculosis and Tetanus toxin.

43. The method of claim 40, wherein said pathogen is a protozoan.

44. The method of claim 43, wherein said protozoan is selected from the group consisting of Plasmodium falciparum, Plasmodium vivax, Toxoplasma gondii, Trypanosoma rangeli, Trypanosoma cruzi, Trypanosoma rhodesiensei, Trypanosoma brucei, Schistosoma mansoni, Schistosoma japanicum, Babesia bovis, Elmeria tenella, Onchocerca volvulus, Leishmania tropica, Trichinella spiralis, Onchocerca volvulus, Theileria parva, Taenia hydatigena, Taenia ovis, Taenia saginata, Echinococcus granulosus and Mesocestoides corti.

45. The method of claim 40, wherein said parasite is a helminth or a malarial parasite.

46. The method of claim 40, wherein said bacterium is mycoplasma.

47. The method of claim 46, wherein said mycoplasma is selected from the group consisting of Mycoplasma arthritidis, M. hyorhinis, M. orale, M. arginini, Acholeplasma laidlawii, M. salivarum, and M. pneumoniae.

48. The method of claim 40, wherein the fungus is selected from the group consisting of Histoplasma capsulatum, Blastomyces dermatitidis, Coccidioides immitis, and species of Candida.

49. The method of claims 9 or 11, wherein the tissue is normal ectopic tissue.

50. The method of claim 49, wherein said normal tissue is tissue from the ovary, thymus, parathyroid, bone marrow, or spleen.

51. The method of claims 9 or 11, wherein said subject is mammalian.

52. The method of claims 9 or 11, wherein said mammalian subject is a human or primate.

53. The method of claim 51, wherein said mammalian subject is selected from the group consisting of rodents, lagamorphs, bovines, ovines, caprines, porcines, equines, canines, felines, domestic fowl, ungulates, and bear.

54. The method of claim 9, wherein the application is for intraoperative diagnosis to identify diseased tissues.

55. The method of claim 9, wherein the application is for endoscopic diagnosis to identify diseased tissues.

56. The method of any one of claims 9-55 wherein a second therapeutic agent is administered before, concurrently, or after the prescribed diagnosis or treatment.

57. The method of claim 56, wherein the second therapeutic agent is a drug, naked antibody, immunomodulator, or antibody conjugate bearing a drug, radioisotope, immunomodulator or toxin.

58. A kit useful for treating or identifying diseased tissues in a subject comprising:
(A) the mutant bispecific antibody of claim 9;
(B) optionally, the clearing agent of claim 9; and
(C) the targetable construct of claim 11.

59. A kit useful for treating or identifying diseased tissues in a subject comprising:
(A) the mutant bispecific antibody of claim 11;
(B) optionally, the clearing agent of claim 11;
(C) the targetable construct of claim 11; and
(D) the prodrug of claim 11.

60. The method of claim 25, wherein said immunomodulator is selected from the group consisting of a cytokine, a stem cell growth factor, a lymphotoxin, a hematopoietic factor, a colony stimulating factor (CSF), an interferon (IFN), erythropoietin, thrombopoietin and a combination thereof.

61. The method of claim 60, wherein said lymphotoxin is tumor necrosis factor (TNF).

62. The method of claim 60, wherein hematopoietic factor is interleukin (IL).

63. The method of claim 60, wherein said colony stimulating factor is granulocyte-colony stimulating factor (G-CSF) or granulocyte macrophage-colony stimulating factor (GM-CSF).

64. The method of claim 60, wherein said interferon is interferon-α, -β or -γ.

65. The method of claim 60, wherein said stem cell growth factor is S1 factor.

66. The method of claim 25, wherein said immunomodulator is IL-1, IL-2, IL-3, IL-6, IL-10, IL-12, IL-18, interferon-γ, TNF-α or a combination thereof.

67. The method of claim 38, wherein said neurological disease is Alzheimer's Disease.


SEQUENCE LISTING (18 sequences)

SEQ ID NO: 1 - DNA, 1407 bp, Homo sapiens
  CDS (1)..(1404)
atgggatggagctgtatcatcctcttcttggtagcaacagctacaggtgtccactccgaggtccaactggtggagagcggtggaggtgttgtgcaacctggccggtccctgcgcctgtcctgctccgcatctggcttcgatttcaccacatattggatgagttgggtgagacaggcacctggaaaaggtcttgagtggattggagaaattcatccagatagcagtacgattaactatgcgccgtctctaaaggatagatttacaatatcgcgagacaacgccaagaacacattgttcctgcaaatggacagcctgagacccgaagacaccggggtctatttttgtgcaagcctttacttcggcttcccctggtttgcttattggggccaagggaccccggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 2 - protein, 468 aa, Homo sapiens
MGWSCIILFLVATATGVHSEVQLVESGGGVVQPGRSLRLSCSASGFDFTTYWMSWVRQAPGKGLEWIGEIHPDSSTINYAPSLKDRFTISRDNAKNTLFLQMDSLRPEDTGVYFCASLYFGFPWFAYWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - DNA, 699 bp, Homo sapiens
  CDS (1)..(696)
atgggatggagctgtatcatcctcttcttggtagcaacagctacaggtgtccactccgacatccagctgacccagagcccaagcagcctgagcgccagcgtgggtgacagagtgaccatcacctgtaaggccagtcaggatgtgggtacttctgtagcctggtaccagcagaagccaggtaaggctccaaagctgctgatctactggacatccacccggcacactggtgtgccaagcagattcagcggtagcggtagcggtaccgacttcaccttcaccatcagcagcctccagccagaggacatcgccacctactactgccagcaatatagcctctatcggtcgttcggccaagggaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 4 - protein, 232 aa, Homo sapiens
MGWSCIILFLVATATGVHSDIQLTQSPSSLSASVGDRVTITCKASQDVGTSVAWYQQKPGKAPKLLIYWTSTRHTGVPSRFSGSGSGTDFTFTISSLQPEDIATYYCQQYSLYRSFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 4 aa, Artificial Sequence
  Description of Artificial Sequence Synthetic peptide
FKYK

SEQ ID NO: 6 - protein, 4 aa, Artificial Sequence
  Description of Artificial Sequence Synthetic peptide
KYKK

SEQ ID NO: 7 - protein, 15 aa, Artificial Sequence
  Description of Artificial Sequence Linker peptide
GGGGSGGGGSGGGGS

SEQ ID NO: 8 - protein, 4 aa, Artificial Sequence
  Description of Artificial Sequence Linker peptide
GGGS

SEQ ID NO: 9 - protein, 5 aa, Artificial Sequence
  Description of Artificial Sequence Linker peptide
GGGGS

SEQ ID NO: 10 - DNA, 56 bp, Artificial Sequence
  Description of Artificial Sequence Synthetic oligonucleotide
ttctctctgcagagcccaaatcttgtggtggcggttcacagctggttgtgactcag

SEQ ID NO: 11 - DNA, 49 bp, Artificial Sequence
  Description of Artificial Sequence Synthetic oligonucleotide
agcctccgcctcctgatccgccacctcctaagatcttcagtttggttcc

SEQ ID NO: 12 - DNA, 35 bp, Artificial Sequence
  Description of Artificial Sequence Synthetic oligonucleotide
ccggaggcggtgggagtgaggtgaaactgcaggag

SEQ ID NO: 13 - DNA, 43 bp, Artificial Sequence
  Description of Artificial Sequence Synthetic oligonucleotide
aaccttgagctcggccgtcgcactcatgaggagacggtgaccg

SEQ ID NO: 14 - DNA, 30 bp, Artificial Sequence
  Description of Artificial Sequence Synthetic oligonucleotide
tccccgggtaaaggtggcggttcacagctg

SEQ ID NO: 15 - DNA, 17 bp, Artificial Sequence
  Description of Artificial Sequence Synthetic oligonucleotide
gagctcggccgtcgcac

SEQ ID NO: 16 - protein, 11 aa, Homo sapiens
KDTLMISRTPE

SEQ ID NO: 17 - protein, 11 aa, Artificial Sequence
  Description of Artificial Sequence Synthetic mutated peptide sequence
KDTLMASRTPE

SEQ ID NO: 18 - DNA, 33 bp, Artificial Sequence
  Description of Artificial Sequence Synthetic oligonucleotide
aaggacaccctcatggctagccggacccctgag